(19) United States
(12) Patent Application Publication
(10) Pub. No.: US 2015/0031964 A1
(43) Pub. Date: Jan. 29, 2015

(54) PHYSIOLOGICAL SIGNAL DETECTING DEVICE AND SYSTEM

(71) Applicant: ACLARIS MEDICAL, LLC, Falcon Heights, MN (US)

(72) Inventors: Mark James Bly, Falcon Heights, MN (US); Scott Mazar, Woodbury, MN (US); Kevin Daniel Ruda, St. Paul, MN (US)

(73) Assignee: ACLARIS MEDICAL, LLC, Falcon Heights, MN (US)

(21) Appl. No.: 14/380,553

(22) PCT Filed: Feb. 22, 2013
(86) PCT No.: PCT/US2013/027300

§ 371 Date: Aug. 22, 2014

Related U.S. Application Data
(60) Provisional application No. 61/601,577, filed on Feb. 22, 2012.

(51) Int. Cl. A61B 5/16; A61B 5/00; A61B 5/0205

(52) U.S. Cl. CPC A61B 5/165; A61B 5/0205; A61B 5/7278; A61B 5/02405
USPC 600/301; 600/300

(57) ABSTRACT

A device configured to detect, measure, and/or monitor physiological signals of a mammal. The device and system can detect a pulse and/or skin bioimpedance of a mammal and determine one or more physiological parameters based on the detected pulse and/or dermal bioimpedance. The device and system converts one or more physiological signals detected by the one or more sensors into one or more physiological parameters and stores the physiological parameters as electronic data, the electronic data being related to a physiological condition of the mammal.

### PRIORITY INFORMATION

This application claims the benefit of priority of U.S. Provisional Application No. 61/601,577 filed on Feb. 22, 2012, which is hereby incorporated by reference in its entirety.

### FIELD

This disclosure is directed to a device, a method, and a system for detecting, measuring, and/or monitoring physiological signals of a mammal.

### BACKGROUND

Perceiving physiological signals from one's own body and then understanding the meaning of the physiological signals is a difficult process for many people. Various physiological signals can be detected and measured to determine various physiological parameters. Doing so, however, can be a complex process requiring multiple devices and/or people.

### SUMMARY

An embodiment of a physiological signal detecting device comprises a sensor that detects a physiological signal, the sensor connected to a sensor decoupling mechanism that reduces noise in the physiological signal detected by the sensor, a processor that receives the physiological signal from the sensor, and converts the physiological signal to a physiological parameter.

An embodiment of the physiological signal detecting device has the sensor configured to be positioned above an artery of a mammal, the processor configured to determine the position of the sensor relative to the artery of the mammal and output information regarding the position of the sensor relative to the artery of the mammal based on the received physiological signal.

An embodiment of the sensor decoupling mechanism includes a rigid portion configured to be positioned lateral to the artery. In an embodiment, the position of the rigid portion does not affect a blood flow through the artery. In an embodiment, the position of the rigid portion minimally affects the blood flow through the artery.

An embodiment of the physiological signal detecting device comprises a multi-point electrodermal activity sensor having at least two alternating current (AC) driving electrodes, and at least two voltage sensing electrodes, a processor which receives a physiological signal from the multi-point electrodermal activity sensor, and converts the physiological signal to a physiological parameter. The embodiment of the physiological signal detecting device can also include a display which displays a user interface and the physiological parameter. In an embodiment, the physiological signal includes a sympathetic response.

An embodiment of the physiological signal detecting device comprises a sensor that detects a physiological signal, a processor that receives the physiological signal from the sensor, processes the physiological signal to reduce artifact signals that are unrelated to the physiological signal, generates a processed physiological data, and converts the processed physiological data to a physiological parameter.

An embodiment of the physiological signal detecting device comprises a sensor that is a pulse sensor.

In an embodiment of the physiological signal detecting device, the physiological signal includes a pulse.

An embodiment of the physiological signal detecting device comprises a display in communication with the processor, and displays a user interface and the physiological parameter.

In an embodiment of the physiological signal detecting device, the processor is configured to determine a second physiological parameter from the physiological parameter.

In an embodiment of the physiological signal detecting device, the processor is configured to determine a stress level based on the physiological parameter, and the display can display the stress level.

An embodiment of the physiological signal detecting device comprises a position sensor in communication with the processor for determining a position of the device.

An embodiment of the physiological signal detecting device comprises an accelerometer in communication with the processor for determining an orientation of the device and/or movement of the device.

An embodiment of the physiological signal detecting device comprises a reed switch for activating the device based on a detected magnetic field.

An embodiment of the physiological signal detecting device comprises a plurality of pressure sensors that detect pulse signals, a processor that receives the pulse signals from the pressure sensors, converts the pulse signals to a physiological parameter, and a display in communication with the processor, and displays a user interface and the physiological parameter.

An embodiment of the physiological signal detecting device includes a network interface connected to the processor for communicating data to another device.

An embodiment of a physiological signal monitoring system comprises any one or more of the embodiments of the physiological signal detecting device described herein, and an auxiliary device. The auxiliary device includes a network interface for communicating with the physiological signal detecting device to receive data related to the physiological signal, and a processor for processing the physiological signal to converted data, and outputting the converted data.

### BRIEF DESCRIPTION OF THE DRAWINGS

FIG. 1 is a schematic diagram of an embodiment of the system for detecting, monitoring, and/or measuring one or more physiological signals.

FIG. 2 is a schematic diagram of an embodiment of the system for detecting, monitoring, and/or measuring one or more physiological signals.

FIG. 3 is a schematic diagram of an embodiment of the system for detecting, monitoring, and/or measuring one or more physiological signals.

FIG. 4 is a schematic diagram of an embodiment of the system for detecting, monitoring, and/or measuring one or more physiological signals.

FIG. 5 is a schematic diagram of an embodiment of the system for detecting, monitoring, and/or measuring one or more physiological signals.

FIG. 6 is a flow diagram of an embodiment of a method for detecting, measuring, and/or monitoring one or more physiological signals.

FIG. 7 is a flow chart of an embodiment of a monitoring and feedback algorithm for a method for detecting, measuring, and/or monitoring one or more physiological signals.

FIG. 8 is a schematic diagram of an embodiment of a physiological signal detecting device.

FIG. 9 is an example of a graph showing a pulse waveform illustrated from pulse signals.

FIG. 10 is a cutaway side view of an embodiment of a physiological signal detecting device.

FIG. 11 is a cutaway side view of an embodiment of a physiological signal detecting device.

FIG. 12 is a cutaway side view of an embodiment of a physiological signal detecting device.

FIG. 13 is a close up view of an embodiment of a pressure sensor section of a physiological signal detecting device.

FIGS. 14 and 15 show embodiments of an X-shaped spring for an embodiment of a physiological signal detecting device.

FIG. 16 is a flow chart of an embodiment of the signal processing algorithm of a method for detecting, measuring, and/or monitoring one or more physiological signals.

FIG. 17 is a flow chart of an embodiment of the signal processing algorithm of a method for detecting, measuring, and/or monitoring one or more physiological signals.

FIG. 18 is a cutaway side view of the positioning of the physiological signal detecting device.

FIGS. 19-23 are examples of shapes of the electrodes for embodiments of a multi-point electrodermal activity sensor.

FIGS. 24 and 25 are perspective views of an embodiment of a physiological signal detecting device.

FIG. 26 is a flow chart of an embodiment of an algorithm of a method for detecting, measuring, and/or monitoring one or more physiological signals.

FIG. 27 is a flow chart of an embodiment of an algorithm of a method for detecting, measuring, and/or monitoring one or more physiological signals.

FIG. 28 is a flow chart of an embodiment of an algorithm of a method for detecting, measuring, and/or monitoring one or more physiological signals.

FIG. 29 is a flow chart of an embodiment of an algorithm of a method for detecting, measuring, and/or monitoring one or more physiological signals.

### DETAILED DESCRIPTION

The present invention may be further understood with reference to the following description and the appended drawings, wherein like elements are referred to with the same reference numerals.

A system, a method, and a device disclosed herein are directed towards detecting, measuring, and/or monitoring physiological signals of a mammal. Physiological signals are generated by a living mammal by functioning physiological systems of the mammal. For example, a blood flowing through an artery is an example of a physiological signal which can be detected. Physiological parameters are measurable features that are related to the physiological signals. For example, heart rate is a physiological parameter which can be measured by detecting the physiological signal of the blood flow through an artery. One type of a physiological signal can be used to derive one or more physiological parameters. One or more types of physiological signals can be used to derive one or more physiological parameters. Further, a physiological parameter can be derived from one or more other physiological parameters. The embodiments described herein are directed towards detecting one or more physiological signals of the mammal, converting the detected physiological signal(s) into one or more physiological parameters, converting one or more physiological parameters into another one or more physiological parameters, and/or storing the physiological signal and/or parameter in a computer readable memory as electronic data. Accordingly, the embodiments described herein can electronically store information related to the physiological parameters detected by one or more sensors that detect physiological signals of the mammal.

For example, embodiments described herein can detect a pulse (a physiological signal) of a mammal and determine one or more physiological parameters of the mammal. Examples of the physiological parameters which can be determined from the pulse signal include heart rate (HR), heart rate variability (HRV), standard deviation of normal sinus to normal sinus (SDNN), standard deviation of the averages of normal sinus to normal sinus (SDANN), blood pressure (BP), pulse wave velocity (PWV), respiratory rate (RR), respiratory rate variability (RRV), tidal volume (V_{T}), tidal volume variability (TVᵥₐᵣ), minute ventilation (MV), stroke volume (SV), cardiac output (CO), and cardiac index (CI). For example, multiple sensors may be needed to determine some of the physiological parameters. For example, two pulse sensors may be needed for measuring PWV.

Various physiological parameters (e.g., heart rate, heart rate variability, blood pressure, electrodermal activity, respiratory rate, skin temperature, etc.) of the mammal can provide information regarding mental, emotional, and/or physical state of the mammal.

The term electrodermal activity is used herein to include both a tonic component (level) (which includes skin conductance response (SCR), skin conductance level (SCL), skin impedance level, skin admittance level, etc.), a phasic component (response) (which includes skin conductance response, galvanic skin reflex, galvanic skin response (GSR), electrodermal response (EDR), skin impedance response, skin admittance response, psychogalvanic reflex (PGR), etc.), capacitive portion (susceptance), and phase angle.

Advantageously, embodiments of the system, the method, and the device disclosed herein can contribute to wellbeing of a mammal by detecting changes in the physiological signals of the mammal.

For example, self-awareness of person's mental, emotional, and physical states are not easily discernible. Being unaware or not understanding the mental, emotional, and physical states can lead to negative consequences both for the person experiencing the state and for others associated with the person. The embodiments disclosed herein can contribute to wellbeing of a person by providing a way for monitoring one or more physiological signals of a person that are related to the person's mental, emotional, and physical states.

For example, the embodiments disclosed herein can contribute to wellbeing of a person suffering from posttraumatic stress disorder (PTSD). The person suffering from PTSD is often not self-aware of his or her state of hyperarousal or a degree thereof. The embodiments disclosed herein can aid in perceiving the hyperarousal symptoms by detecting changes in one or more physiological signals of the person. Thus, the embodiments disclosed herein can assist in diagnosing, evaluating, and/or treating PTSD. Psychophysiological data could be used in conjunction with subjective diagnostic assessments to evaluate and guide therapy, reinforce progress, and motivate patients to continue treatment. In prolonged exposure therapy, the level of psychophysiological reactivity to imaginal and in-vivo exposures during sessions and homework could assist the therapist with constructing an initial hierarchy, identifying and focusing on hot spots, selecting homework, evaluating therapy progress, adjusting the hierarchy, and determining when to move on. A smartphone/tablet app with an in-vivo hierarchy and recorded imaginal exposures could use psychophysiological input along with ecological and survey input to adapt exposure type, duration, and frequency so as to optimize individual outcomes. In cognitive processing therapy, psychophysiological data collected both during sessions and homework could help the therapist identify and focus on stuck points, select homework, and assess improvement. Ambulatory psychophysiological data could provide additional information regarding chronic hyperarousal, situational physiological reactivity, and sleep disturbance. Psychophysiological data could also be used in conjunction with smartphone-based ecological momentary assessment, which could be triggered periodically, randomly, or eventually using physiological thresholds. Physioloigcal data could be useful in research to compare modes of psychotherapy, evaluate differences between responders and nonresponders, and better understand autonomic hyperarousal during sleep.

For example, the embodiments disclosed herein can contribute to wellbeing of a person suffering from a panic disorder. The person suffering from the panic disorder may feel that he or she is unable to self-predict when he or she will experience a panic attack because of the unpredictable nature of the panic attacks. The embodiments disclosed herein can assist in predicting when the sufferer will experience a panic attack before the full occurrence of the panic attack by detecting changes in one or more physiological signals of the person.

For example, the embodiments disclosed herein can contribute to wellbeing of a person suffering from depression. The person suffering from depression who is on the verge of a depressive episode may experience fatigue and lack of motivation. Often, the feeling of lack of motivation is self-diagnosed by the sufferer as a direct result of the fatigue. While these depressive episodes may appear to arise acutely, preliminary physiological signs can exist unbeknownst to the sufferer. Being aware of these preliminary physiological signs can lead to actions which can lead to preventing the depressive episode. The embodiments disclosed herein can detect, monitor, and analyze one or more physiological signals of the person that are associated with the depressive episode.

For example, the embodiments disclosed herein can contribute to wellbeing of a person suffering from general stress. General stress can go unnoticed until the stress results in physical symptoms (e.g., increased level of stomach acid, lower back pain, difficulty in sleeping, bruxism, poor sexual performance, social withdrawal, impact on relationships, mood swings, mental breakdown, etc.). Due to difficulty in self-recognizing the rising levels of stress, many people do not seek a way of reducing stress. Even when a person seeks a way of reducing stress, it is often not until an unfortunate and significant event has occurred. Often, the unfortunate significant event includes emotional damage and/or physical damage. The embodiments disclosed herein can help to detect, monitor, and analyze one or more physiological signals of the person that are associated with general stress so that these unfortunate significant events can be avoided.

For example, the embodiments disclosed herein can contribute to wellbeing of a person suffering from not having restful sleep. For some people, the reason for not having restful sleep is not clearly understood because they have difficulty remembering what happened during the sleeping period. The embodiments disclosed herein can detect, monitor, and store one or more physiological signals of a person that occur during the sleep period to a computer readable memory as physiological data so that the physiological data can be analyzed to better understand the reason for not having restful sleep.

For example, the embodiments disclosed herein can contribute to wellbeing of a person suffering from drug addiction. The person seeking treatment for drug abuse may relapse because he or she is not self-aware of craving that are slowly building up until the craving becomes too difficult to manage. The embodiments disclosed herein can help to detect, monitor, and analyze one or more physiological signals that are associated with the craving (or change in the level of the craving) so that the person can seek help before the craving becomes too difficult to manage.

For example, the embodiments disclosed herein can contribute to proper delivery of medication to a person. An accidental overdose (or other improper delivery of medication) is often discovered too late. Often, the late discovery is because the person who took the improper amount of medication failed to recognize the physiological signals and/or changes in the physiological signals as being caused by the improper amount of the medication in the person's system. The embodiments disclosed herein can help determine an improper delivery of a medication by detecting and/or monitoring one or more physiological signals and/or changes in the one or more physiological signals of the person who took the medication. The embodiments can help in determining that a person did not take an improper amount of a medication. The embodiments can help in determining that a person did take an improper amount of a medication. The embodiments can help in determining that a person did take an improper amount of a medication before the person suffers irreversible damage.

For example, the embodiments disclosed herein can contribute to wellbeing of a person who has difficulty managing his or her weight, by detecting, monitoring, and/or analyzing one or more physiological signals related to the amount of calories being consumed and/or burnt.

For example, the embodiments disclosed herein can contribute to wellbeing of a patient, by detecting, monitoring, and/or analyzing one or more physiological signals of the hospital patient, especially when that patient has difficulty communicating (e.g., an unconscious patient) to the hospital staff. For example, the embodiments disclosed herein can be worn by a monitored person to monitor a disease (e.g., heart failure, COPD, diabetes, hypertension, obesity, kidney failure, etc.). For example, the embodiments disclosed herein can be worn by a monitored person for general vital signs monitoring or can be configured to monitor a particular disease or condition (e.g., heart failure, recovery from heart surgery, pneumonia, COPD, ambulatory blood pressure monitoring, etc.).

The embodiments described herein can be used as an ambulatory monitoring device and system. For example, the device and/or system can detect decrease in pulse pressure amplitude (PP), decrease in pulse pressure width, decrease in area under pulse pressure waveform, increase in pulse pressure oscillations, increase in heart rate (HR), increase in respiratory rate (RR), and/or decrease in heart rate variability (HRV).

Such device and system may be used in an emergency room of a hospital (or elsewhere) to monitor a person requiring medical triage. For example, the device and/or system can detect hypovolemia (a state of decreased blood volume) caused by hemorrhaging by monitoring one or more of the following: decrease in pulse pressure amplitude (PP), decrease in pulse pressure width, decrease in area under pulse pressure waveform, increase in pulse pressure oscillations, increase in heart rate (HR), increase in respiratory rate (RR), and decrease in heart rate variability (HRV).

Further, such device and system may be used by a person who engages in perilous activity (e.g., scuba diving, firefighting, police, mining, spelunking, etc.). Under these conditions, the monitoring device and system allow the monitored person's physiological condition to be monitored by another device and/or a person.

The embodiments disclosed herein can contribute to wellbeing of a person who cannot communicate well with other persons. For example, an autistic person, an infant, and/or a child may have difficulty communicating how they feel, especially when they feel stressed. The embodiments disclosed herein can contribute to wellbeing of such persons by detecting, monitoring, and/or measuring one or more physiological signals (or changes of the signals) that manifest from the person's mental state (or a change in the mental state) which can be used to communicate the persons' emotional state to another device and/or another person.

For example, the embodiments disclosed herein can contribute to analyzing physical performance of a person (e.g., an athlete, public service provider such as police, fireman, military personnel). The embodiments can be used to detect, monitor, and store one or more physiological signals during (as well as before and after) physical exertion to provide feedback and analysis to improve optimal performance and endurance, with the embodiments being able to provide quick feedback when the person's exertion is at dangerous levels. Thus, the embodiments can help in preventing overexertion which can lead to permanent damage or even death related to overexertion.

For example, the embodiments disclosed herein can receive physiological feedback from a person who is participating in leisure activities, such as playing a computer game, or receiving a particular service, or enjoying a particular event. The detection of the person's one or more physiological signals related to the person's emotional state and/or mental state can assist in capturing data related to how the person truly feels and/or thinks about any of the activities. It can be very useful to know the true, perhaps even subconscious, feelings of the person regarding a particular person, place, and/or thing.

The embodiments disclosed herein can be used for providing market feedback or enhancing survey/polling data, by monitoring a person's physiological signals and parameters as the monitored person experiences experiencing one or more activities (e.g., shopping for a product, consuming a service, purchasing a product, eating, watching television, watching a movie, listening to music, chatting with a person, dating, etc), in one or more surroundings (e.g. at home, in a theatre, in a movie theatre, in a store, in a restaurant, in a spa, at an amusement park, online, etc.) to measure whether the monitored person likes or dislikes a person, place, thing, etc. Baseline data may be collected related to one or more liked or disliked persons, places or things to determine the baseline physiological data associated with the like or dislike so that the system can recognize when a monitored person likes or dislikes a person, place or thing in the future and make suggestions (e.g., alternative songs, restaurants, friends, dates, etc.) based on the liking or disliking. The embodiment can include an index of like or dislike. The index may be a continuous scale (e.g., from −10 to 10, from 0 to 1000, etc.) indicating a gradient from least to most like. Based on the collected data of the monitored person, a prediction may be made for various persons, places, things, etc. Further, based on the collected data from similar persons, places, things, etc. and/or responses to questions, and/or the currently measured physiological data, a prediction may be made for various persons, places, things, etc. For example, a preferred song may be predicted based on preferences from previously entered input data and/or previously recorded physiological data used in conjunction with current input data (e.g., mood, etc.) and/or current physiological data. The resulting indices, statuses, or predictions, may trigger feedback data that may be provided in the form of alerts, alarms, messages, etc. For example, the monitored person may be provided with feedback data that he is not liking a person, place, thing, etc. or that he is not excited or aroused by a person, place, thing, etc. so that the monitored person chooses an alternative person, place, thing, etc. The feedback data may take the form of a light or message or other visual indicator on the physiological signal detecting device or on an auxiliary system. The feedback data may also take the form of physical feedback (e.g., vibration, pressure, etc.). The feedback data may include instructions for the monitored person, monitoring administrator (e.g., market researcher, etc.), or other person (e.g. friend, gift purchaser, online friend or connection, etc.). For example, feedback data may be given as to music, television programming, movies, gifts, friends, dates, etc., that the monitored person may like or dislike. Data (e.g., raw data, derived data, indices, statuses, predictions, alerts, alarms, messages, instructions, etc.) may be communicated to the monitored person, monitoring administrator, or other person. The physiological signal detecting device or an auxiliary device may provide feedback data to an auxiliary system to change its output. For example, the physiological signal detecting device and/or auxiliary device may provide feedback data to an auxiliary system that is an MP3 player or stereo such that the music is initiated, stopped, selected and/or changes based on the feedback data from the physiological signal detecting device. An embodiment of the system includes multiple physiological signal detecting devices to gather data from multiple persons. For example, multiple persons using an online social network may be monitored such that an online music provider alters the play list based on feedback data from the multiple persons. In another example, market research may be conducted across multiple persons being monitored online and/or while experiencing the natural world (e.g. for a particular movie, restaurant, product, store, etc.). The liking or disliking of different persons, places, things, etc. may be displayed to show how persons, places, things, etc. rank against each other. For example, the relative performance of persons, places, things, etc. may be displayed online and/or on individual physiological signal detecting devices and/or on auxiliary devices.

The embodiments disclosed herein can contribute to wellbeing of a person by detecting, monitoring, and/or measuring one or more physiological signals (or changes of the signals) that manifest from the person's mental state (or a change in the mental state) which can be stored to a computer readable memory as electronic data, displayed on a computer, and analyzed for interpretation of the data.

An embodiment of the system includes a physiological signal detecting device and an auxiliary device. Examples of the physiological signal detecting device include a wearable device having a strap and at least one sensor configured to detect a physiological signal. Examples of the auxiliary device include a Personal Computer (PC), a server, a cloud computing environment, a tablet, a smart phone, and/or any combination thereof. The auxiliary device can include one or more sensors (accelerometer, ambient temperature, etc). A processor on the auxiliary device may process data not only related to one or more sensor devices but also from sensors on one or more auxiliary devices.

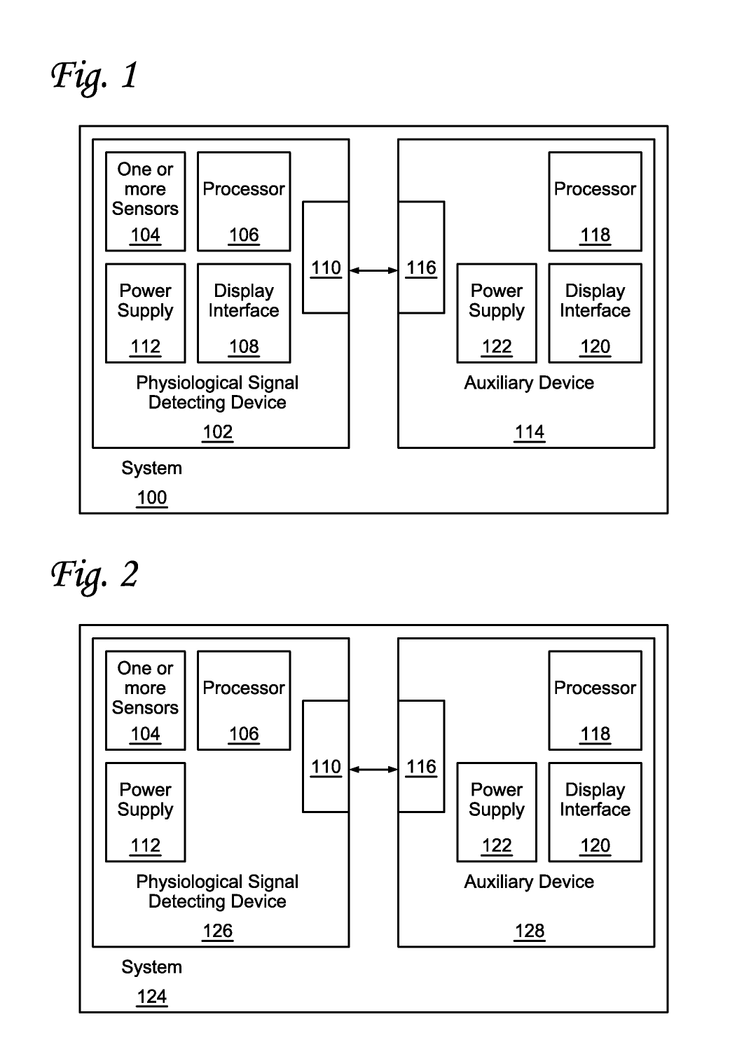

FIG. 1 is a schematic diagram of an embodiment of the system 100 for detecting, monitoring, and/or measuring one or more physiological signals (or changes of the signals) of a mammal. The system 100 is configured for storing to a computer readable memory the one or more physiological signals as electronic data, displaying the data on a computer screen, and/or analyzing the data for interpretation of the data.

The system 100 may also be configured for converting the one or more physiological signals into one or more physiological parameters, and storing the physiological parameters into the computer readable memory as data.

The system 100 may also be configured for converting one or more physiological parameter into another one or more physiological parameters, and storing the physiological parameters into the computer readable memory as data.

The system 100 includes a physiological signal detecting device 102 which includes one or more sensors 104 for detecting one or more physiological signals of the mammal. The physiological signal detecting device 102 of the system 100 includes a processor 106 which receives the physiological signals detected by the one or more sensors 104, and processes the detected physiological signals. The term “processor” is used herein to include (or in communication with) a memory component that stores computer readable instructions which is executable for performing a method according to an algorithm of the computer readable instructions. The memory component of the processor 106 can store data resulting from the execution of the computer readable instructions. The term “processor” includes a computing processor connected to and/or in communication with a “memory” that stores computer readable electronic data. Examples of memory include random access memory (RAM), read only memory (ROM), buffer memory, magnetic medium storage device, optical storage device, flash memory, etc. The memory component of the processor 106 stores the data resulting from the physiological signals received from the one or more sensors 104. Examples of the sensor include a pressure sensor, a force sensor, a strain sensor (e.g. resistive or piezoresistive strain sensor including metallic, semiconductor, or conductive polymer strain gauge), a flow rate sensor, an optical sensor (e.g. infrared sensor), an ultrasonic sensor, an acoustic sensor (e.g. microphone), a radar, a Doppler radar, an accelerometer, a gyroscope, an impedance/conductance sensor, a 4-point impedance sensor, a voltage sensor, two or more electrodes used to collect an electrical voltage signal (ECG, EEG, EMG, etc.), a piezoelectric sensor, a piezojunction sensor, a capacitive sensor, a tunneling sensor, a photoemissive sensor, a photoconductive sensor, a junction-based photodetector (e.g. photodiode, phototransistor, etc.), a capacitive photosensor, a pyroelectric sensor, a bolometer, a thermopile, a Peltier module, a thermoresistive sensor (e.g. a thin-film thermoresistor, and a thermistor), a thermocouple, a diode temperature sensor, a transistor temperature sensor, a magnetic sensor (e.g. hall effect sensor), a chemical sensor, a global positioning system (GPS) receiver, at least four electrodes, greater than four electrodes, etc.

The system 100 converts one or more physiological signals detected by the one or more sensors 104 into one or more physiological parameters and stores the physiological parameters as electronic data. Accordingly, the electronic data stored in the processor 106 is related to physiological parameters (e.g., condition) of the monitored mammal. Examples of physiological parameters collected by the system 100 include, but not limited to, pulse waveform, HR, HRV, SDNN, SDANN, PWV, RR, RRV, V_{T}, TVᵥₐᵣ, MV, SV, CO, CI, electrocardiogram (ECG), BP (e.g. systolic blood pressure, diastolic pressure, mean arterial pressure, central arterial pressure, etc.), respiratory sinus arrhythmia (RSA), heart rate variability (HRV), electrodermal activity, bioimpedance (i.e. tissue impedance, impedance plethysmography, impedance tomography), fluid concentration, body composition, body fat %, calories burned, heat flux, body temperature, ambient temperature, activity, movement, posture, muscle tension, muscle relaxation, electromyogram (EMG), electrooculogram (EOG), pulse oximetry, oxygen saturation (e.g. SpO2), carbon dioxide saturation (e.g. SpCO2), glucose concentration or level, electrical brain activity, electroencephalogram (EEG), circadian rhythm, sound, light, location, and any combinations thereof.

The processor 106 can be configured to convert one or more physiological signals detected by the one or more sensors 104 into one or more physiological parameters and store the physiological parameters as electronic data into a memory component of the processor 106 (or to a memory connected to the processor 106). The processor 106 can be configured to convert one or more physiological parameters into one more other physiological parameters and store these physiological parameters as electronic data into a memory component of the processor 106 (or to a memory connected to the processor 106).

For example, the processor 106 can include computer readable instructions that determine multiple respiratory parameters (e.g., RR, RRV, etc.) from externally measured radial artery pulse signals to detect severe respiratory depression. The processor 106 can include computer readable instructions that use one or more of the following example parameters and alerts of a respiratory depression. The example parameters are:
Respiratory rate has dropped below threshold of, for example, 8 or 10 breaths per minute or reduction of >50% from baseline;
Respiratory rate variability exceeds threshold of, for example, increase of more than 150% from baseline;
Tidal volume variability exceeds threshold, for example, increase of more than 150% from baseline;
The rate at which RR drops exceeds threshold;
Expiratory time exceeds threshold, for example, increase of more than 100% from baseline; and/or
Minute ventilation drops below threshold, for example, reduction of more than 50% from baseline.

The physiological signal detecting device 102 of the system 100 includes a display interface 108 for displaying the electronic data and/or a user interface which allows a user to interact with the physiological signal detecting device 102. The interaction includes user input and output for communicating with the computer readable instructions executed by the processor 106. The display interface 108 can include separate components, such as a display component and an interface component. For example, the display component may be one or more light-emitting diodes (LED) or a liquid crystal display (LCD), and the interface component may be a piece of hardware or mechanism, such as, for example, a one or more physical buttons. The display interface 108 may be a single component which has both a display function and an interface function. For example, the display interface 108 may be a LED or LCD display having a touch screen interface. Examples of the display interface 108 also include an electronic paper display, a keypad, a mouse, an electronic stylus, a microphone (for receiving a voice command), and any combinations described above.

The physiological signal detecting device 102 of the system 100 includes a network interface 110 for wired and/or wireless connection for receiving and/or transmission of electronic information to and/or from an auxiliary device 114 of the system 100. The electronic information can include electronic data related to a physiological condition of the monitored mammal, the physiological signals detected by the one or more sensors 104, and/or the physiological parameters of the monitored mammal. The wired and/or wireless connection may be via the internet, a local network, the “cloud” (i.e., cloud computing environment), a direct connection between hardware, etc. Examples of the wired connection include electronic information transfer via a data cable, a USB cable, an Ethernet cable, a HDMI cable, etc. Examples of the wireless connection include electronic information transfer via WiFi, Bluetooth, Zigbee (i.e., mesh network), near field communication (NFC), radio, microwave, infrared, laser, etc. For the operation of the components of the physiological signal detecting device 102, a power supply 112 supplies power to the one or more sensors 104, the processor 106, the display interface 108, and the network interface 110.

The auxiliary device 114 includes a network interface 116 for wired and/or wireless connection for receiving and/or transmission of the electronic information to and/or from the physiological signal detecting device 102. The auxiliary device 114 includes a processor 118 which can receive and process physiological data and display the data on a display interface 120 in textual format and/or graphical format. The display interface 120 can be similar to the display interface 108 described above. The display interface 120 for displaying the electronic data and/or a user interface which allows a user to interact with the auxiliary device 114 and/or the physiological signal detecting device 102. The interaction includes user input and output for communicating with the computer readable instructions executed by the processor 106 and/or the processor 118. The display interface 120 can include separate components, such as a display component and an interface component. For example, the display component may be one or more LEDs or a LCD, and the interface component may be a piece of hardware or mechanism, such as, for example, a one or more physical buttons. The display interface 120 may be a single component which has both a display function and an interface function. For example, the display interface 120 may be a LED or LCD display screen having a touch screen interface. Examples of the display interface 120 also include an electronic paper display, a keypad, a mouse, an electronic stylus, a microphone (for receiving a voice command), and any combinations described above.

The processor 118 of the auxiliary device 114 is configured to convert the physiological signals detected by the one or more sensors 104 into physiological parameters and stores the physiological parameters as electronic data into a memory component of the processor 118. The processor 118 can be configured to convert one type of physiological parameters into another type of physiological parameters according to a relationship algorithm, wherein the relationship algorithm is a part of the computer readable instructions executed by the processor 118. The display interface 120 can display the electronic data and/or a user interface for the auxiliary device 114, which allows a user to interact with the auxiliary device 114 and/or the physiological signal detecting device 102. The interaction includes user input and output for communicating with the computer readable instructions executed by the processor 118 and/or by the processor 106. For the operation of the components of the auxiliary device 114, a power supply 122 supplies power to the processor 118, the display interface 120, and the network interface 116.

FIG. 2 is a schematic diagram of another embodiment of the system 124 for detecting, measuring, and/or monitoring one or more physiological signals of a mammal. The system 124 includes a physiological signal detecting device 126 which includes one or more sensors 104 for detecting one or more physiological signals of the mammal. The physiological signal detecting device 126 includes a processor 106 which receives the physiological signals detected by the one or more sensors 104, and processes the detected physiological signals. The system 124 converts the physiological signals detected by the one or more sensors 104 into physiological parameters and stores the physiological parameters as electronic data. For example, the processor 106 can convert the physiological signals detected by the one or more sensors 104 into physiological parameters and stores the physiological parameters as electronic data into a memory component of the processor 106. The physiological signal detecting device 126 includes a network interface 110 for wired and/or wireless connection for receiving and/or transmission of electronic information to and/or from an auxiliary device 128. For the operation of the components of the physiological signal detecting device 126, a power supply 112 supplies power to the one or more sensors 104, the processor 106, and the network interface 110. The physiological signal detecting device 126 does not include a display interface for displaying a user interface which allows a user to interact with the physiological signal detecting device 126. Detecting the physiological signals is automated by the processor 106 and/or is controlled via the auxiliary device 128 connected to the physiological signal detecting device 126.

The auxiliary device 128 includes a network interface 116 for wired and/or wireless connection for receiving and/or transmission of the electronic information to and/or from the physiological signal detecting device 126. The auxiliary device 128 includes a processor 118 which can receive and process physiological data and display the data on a display interface 120 in textual format and/or graphical format. The processor 118 and the display interface 120 can be configured to control the physiological signal detecting device 126, and/or receive output data from the physiological signal detecting device 126 and display the output data. The display interface 120 can display the electronic data and/or a user interface for the auxiliary device 128, which allows a user to interact with the auxiliary device 128 and/or the physiological signal detecting device 126. The interaction includes user input and output for communicating with the computer readable instructions executed by the processor 118 and/or by the processor 106. For the operation of the components of the auxiliary device 128, a power supply 122 supplies power to the processor 118, the display interface 120, and the network interface 116.

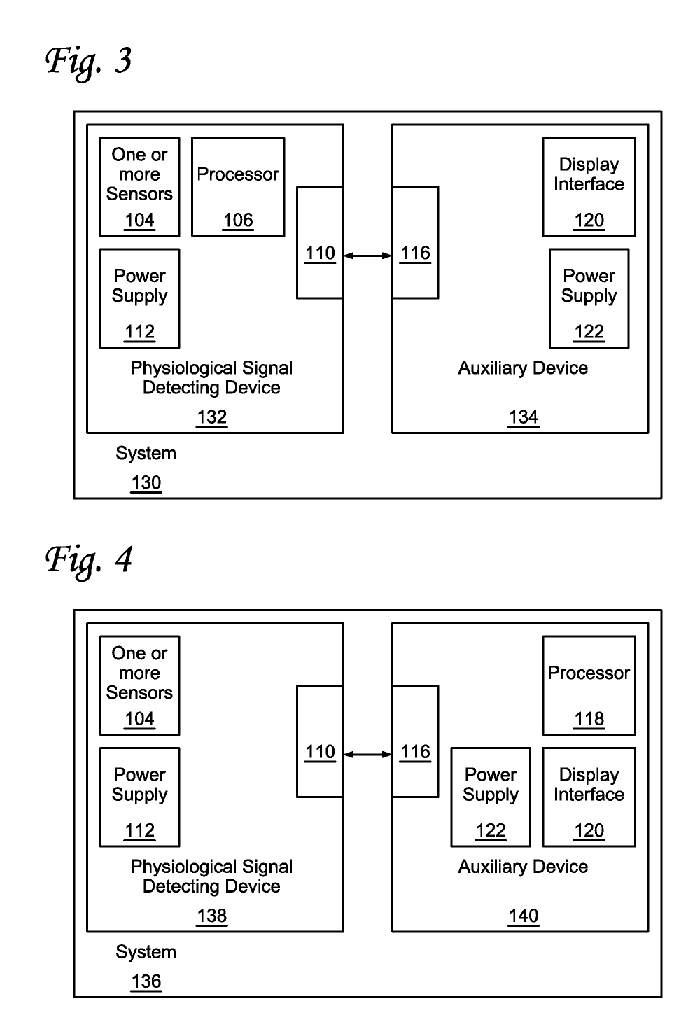

FIG. 3 is a schematic diagram of another embodiment of the system 130 for detecting, measuring, and/or monitoring one or more physiological signals of a mammal. The system 130 includes a physiological signal detecting device 132 which includes one or more sensors 104 for detecting one or more physiological signals of the mammal. The physiological signal detecting device 132 includes a processor 106 which receives the physiological signals detected by the one or more sensors 104, and processes the detected physiological signals. The system 130 converts the physiological signals detected by the one or more sensors 104 into physiological parameters and stores the physiological parameters as electronic data. For example, the processor 106 can convert the physiological signals detected by the one or more sensors 104 into physiological parameters and stores the physiological parameters as electronic data into a memory component of the processor 106. The physiological signal detecting device 132 includes a network interface 110 for wired and/or wireless connection for receiving and/or transmission of electronic information to and/or from an auxiliary device 134. For the operation of the components of the physiological signal detecting device 132, a power supply 112 supplies power to the one or more sensors 104, the processor 106, and the network interface 110. The physiological signal detecting device 132 does not include a display interface for displaying a user interface which allows a user to interact with the physiological signal detecting device 132. Detecting the physiological signals is automated by the processor 106 and/or is controlled via the auxiliary device 134 connected to the physiological signal detecting device 132.

The auxiliary device 134 includes a network interface 116 for wired and/or wireless connection for receiving and/or transmission of the electronic information to and/or from the physiological signal detecting device 132. The auxiliary device 134 does not include a processor that processes the physiological data. Instead, the auxiliary device 134 displays the data received from the physiological signal detecting device 132 directly to a display interface 120. The processor 106 and the display interface 120 are in communication so that they can be used together to control the physiological signal detecting device 132. The display interface 120 can display the electronic data and/or a user interface, which allows a user to interact with the physiological signal detecting device 132. The interaction includes user input and output for communicating with the computer readable instructions executed by the processor 106. For the operation of the components of the auxiliary device 134, a power supply 122 supplies power to the display interface 120, and the network interface 116.

FIG. 4 is a schematic diagram of another embodiment of the system 136 for detecting, measuring, and/or monitoring one or more physiological signals of a mammal. The system 136 converts the physiological signals detected by the one or more sensors 104 into physiological parameters and stores the physiological parameters as electronic data. The system 136 includes a physiological signal detecting device 138 which includes one or more sensors 104 for detecting one or more physiological signals of the mammal. The physiological signal detecting device 138 includes a network interface 110 for wired and/or wireless connection for receiving and/or transmission of electronic information to and/or from an auxiliary device 140. For the operation of the components of the physiological signal detecting device 138, a power supply 112 supplies power to the one or more sensors 104 and the network interface 110. The physiological signal detecting device 138 does not include a processor which receives the physiological signals detected by the one or more sensors 104, and processes the detected physiological signals. Instead, the physiological signal detecting device 138 communicates the raw physiological signal data detected by the one or more sensors 104 to an auxiliary device 140 via the network interface 110. The physiological signal detecting device 138 does not include a display interface for displaying a user interface which allows a user to interact with the physiological signal detecting device 138. Detecting the physiological signals is controlled by a processor 118 of the auxiliary device 140. The auxiliary device 140 includes a network interface 116 for wired and/or wireless connection for receiving and/or transmission of the electronic information to and/or from the physiological signal detecting device 138. The processor 118 can convert the physiological signals detected by the one or more sensors 104 into physiological parameters and stores the physiological parameters as electronic data into a memory component of the processor 118. The processor 118 can process the physiological data and display the data via a display interface 120 in textual format and/or graphical format. The processor 118 and the display interface 120 are configured to control the physiological signal detecting device 138, and for receiving output data from the physiological signal detecting device 138. The display interface 120 can display the electronic data and/or a user interface for the auxiliary device 140, which allows a user to interact with the auxiliary device 140 and the physiological signal detecting device 138. The interaction includes user input and output for communicating with the computer readable instructions executed by the processor 118. For the operation of the components of the auxiliary device 140, a power supply 122 supplies power to the processor 118, the display interface 120, and the network interface 116.

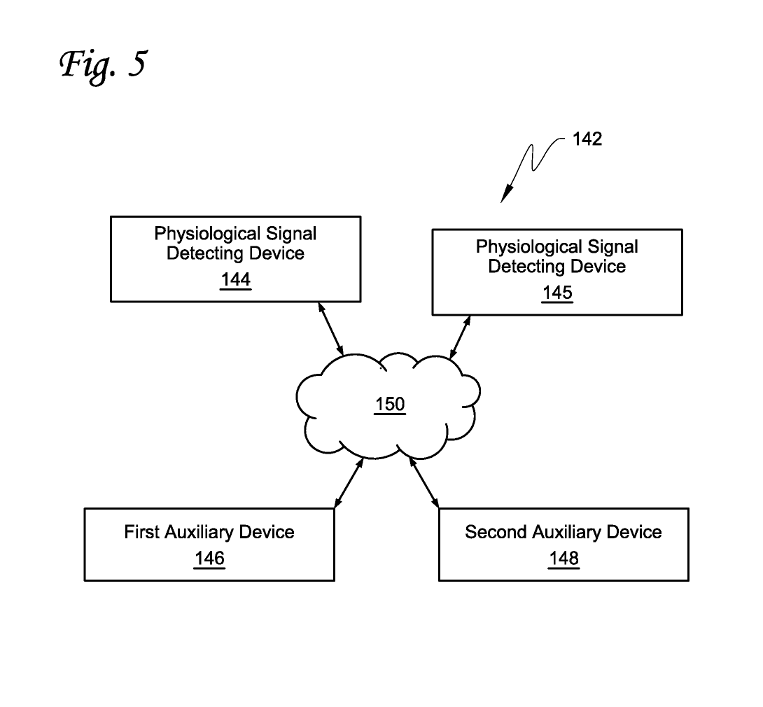

FIG. 5 is a schematic diagram of an embodiment of the system 142 for detecting, measuring, and/or monitoring physiological signals of a mammal. The system 142 includes one or more physiological detecting devices 144, 145, each of which can be, or similar to, the physiological detecting devices 102, 126, 132, 138 shown in FIGS. 1-4. One or more of the physiological detecting devices 144, 145 are connected to one or multiple auxiliary devices 146, 148 for communicating data related to physiological signals detected by the physiological detecting devices 144. Each of the auxiliary devices 146, 148 can be or can be similar to the auxiliary devices 114, 128, 134, 140 shown in FIGS. 1-4. One or more physiological detecting devices 144, 145 and one or multiple auxiliary devices 146, 148 communicate through a network 150. Examples of the network 150 include wired and/or wireless network in a local area network (LAN), the internet, WiFi, Zigbee, NFC, cellular, or any combinations thereof. For example, multiple physiological detecting devices 144 may be placed on a single mammal. Further, a first monitoring person may interact with the first auxiliary device 146 to monitor the mammal wearing the one or more physiological detecting devices 144, 145, while another second monitoring person interacts with the second auxiliary device 148 to monitor the mammal wearing the one or more physiological detecting devices 144, 145.

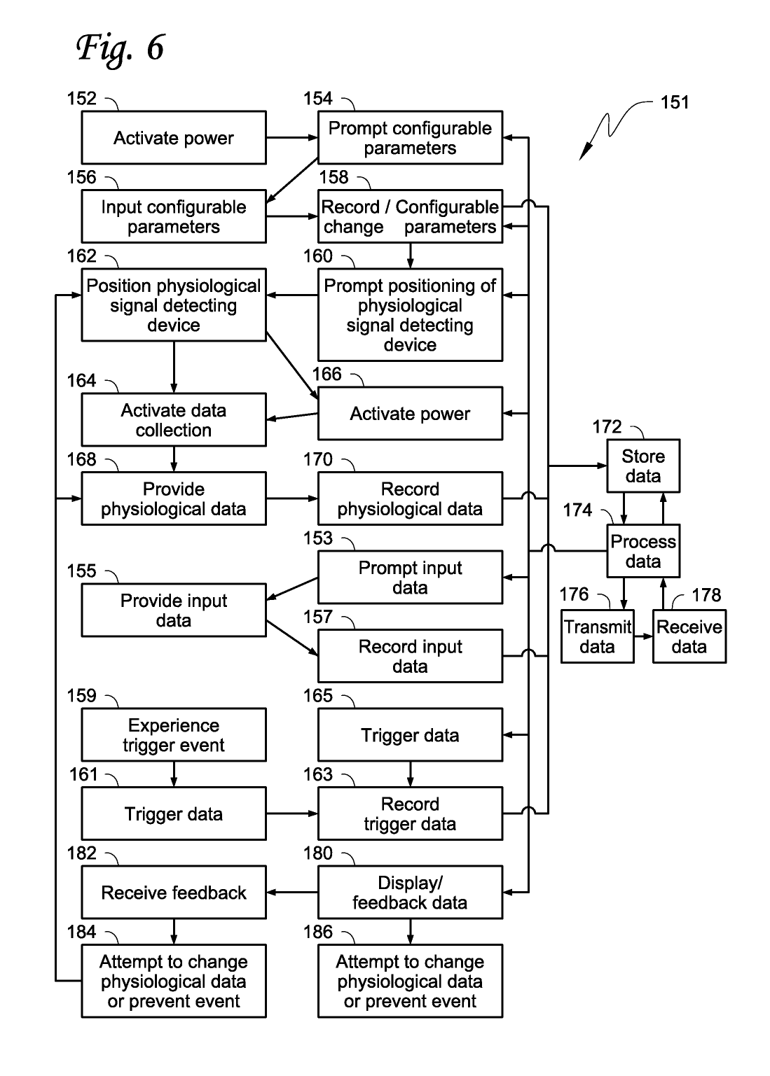

FIG. 6 is a flow diagram of an embodiment of a method 151 for using any of the embodiments of the system disclosed above for detecting, measuring, and/or monitoring one or more physiological signals of a mammal. The method 151 includes turning on the system by activating power 152 to the system. The system provides a user interface via a display interface of the system and prompts the user to configure the system 154. Configuring the system allows the user to input 156 and configure which one or more physiological parameters are to be detected, measured, and/or monitored by the system. Configuring the system may also allow entering data about the monitored person and/or calibration data. The system can store 158 this user input into a computer readable memory component of the system. The user interface prompts 160 the user to position a physiological signal detecting device of the system to a mammal's body. For example, for a human, the physiological signal detecting device may be positioned at a wrist, ankle, upper arm, upper leg, head, chest, or other body part suitable to detect a physiological signal. The system may be configured to automatically detect whether the physiological signal detecting device of the system has been properly positioned to the mammal's body. The system waits for the user to position 162 the physiological signal detecting device to an appropriate location on a body for detecting the physiological signal. The user can activate data collection and physiological signal detection 164 after the physiological signal detecting device has been properly positioned. The system activates necessary components by delivering power to those components 166. The physiological signal detecting device starts detecting 168 a physiological signal of the mammal (e.g., a person) and provides the physiological signal as data to the system. The system converts the physiological signal to one or more physiological parameters and stores 170 the physiological signal(s) and/or physiological parameter(s) as electronic data in computer readable format to a memory component of the system. The system performs necessary functions for performing the detecting, measuring, and/or monitoring the one or more physiological signals by storing 172, processing 174, transmitting 176, and/or receiving 178 electronic data related to the one or more physiological signals. The system displays 180 one or more physiological parameters and/or data related to the detected physiological signal(s). The user can receive 182 the feedback information, and based on the received feedback information, the user may become aware 184 of a physiological condition related to the physiological feedback information so that the user can attempt to change the physiological signal(s) and/or attempt to prevent an event resulting from the physiological signal(s). The system may be configured to attempt 186 to change the physiological signal(s) of the user via an automated process or by alerting another person as an attempt to prevent an event resulting from the physiological signal(s).

The method 151 includes the system prompting the user to input data. When the user inputs the requested data 153, the system records the input data 157.

When the method 151 has a triggering event 159, the system collects trigger data 161 and records the triggered data 163. The system can also generate trigger data 165, which is also recorded as triggered data 163 in the system.

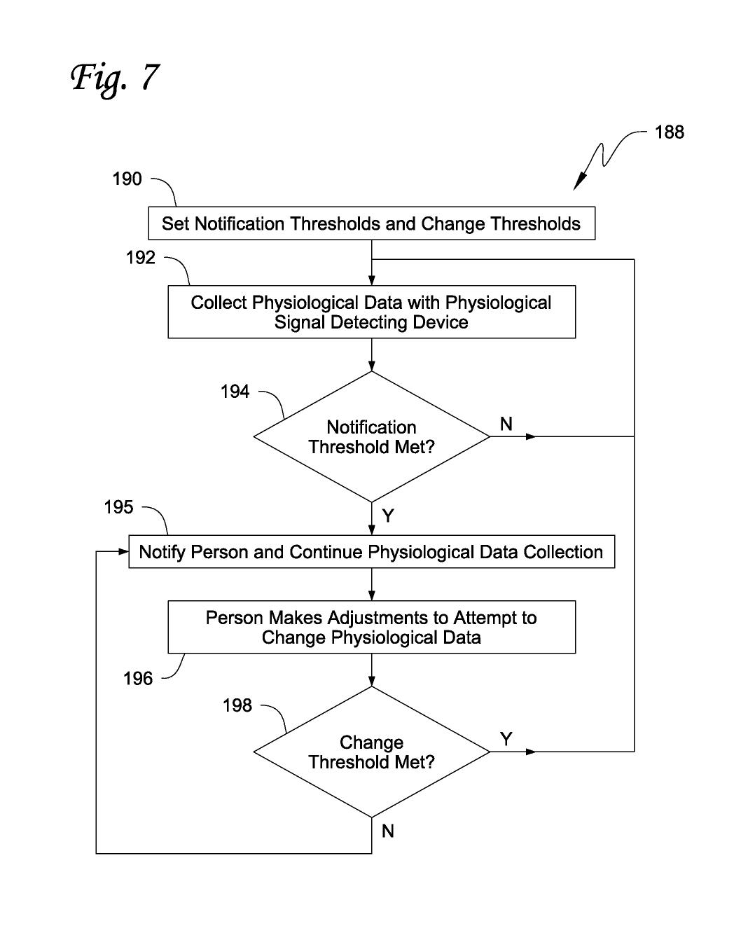

FIG. 7 is a flow chart of an embodiment of a monitoring and feedback algorithm for a process 188 in computer readable instructions which is stored and executed by the processor 106 and/or the processor 118. The process 188 provides the feedback information to the person being monitored with the embodiments of the devices and/or systems described herein. The process 188 includes setting a notification threshold and/or making a change to a set threshold 190, collecting physiological data with the sensor detection device 192, determining whether the collected data meets the notification threshold 194. If the collected data does not meet the notification threshold, then the process 188 loops back to collecting physiological data with the sensor detection device 192. If the collected data does meet the notification threshold, then the processor 106 performs notification to a person (e.g., the person being monitored and/or a health professional) that the collected data has met the notification threshold and continues to collect physiological data 195. The process 188 then waits for the notified person to adjust and/or attempt to change physiological data 196, while continuing the notification and collecting physiological data 195 until a determination has been made that the collected data no longer meets the notification threshold 198. If the collected data no longer meets the notification threshold, then the process 188 returns to collecting physiological data with the sensor detection device 192.

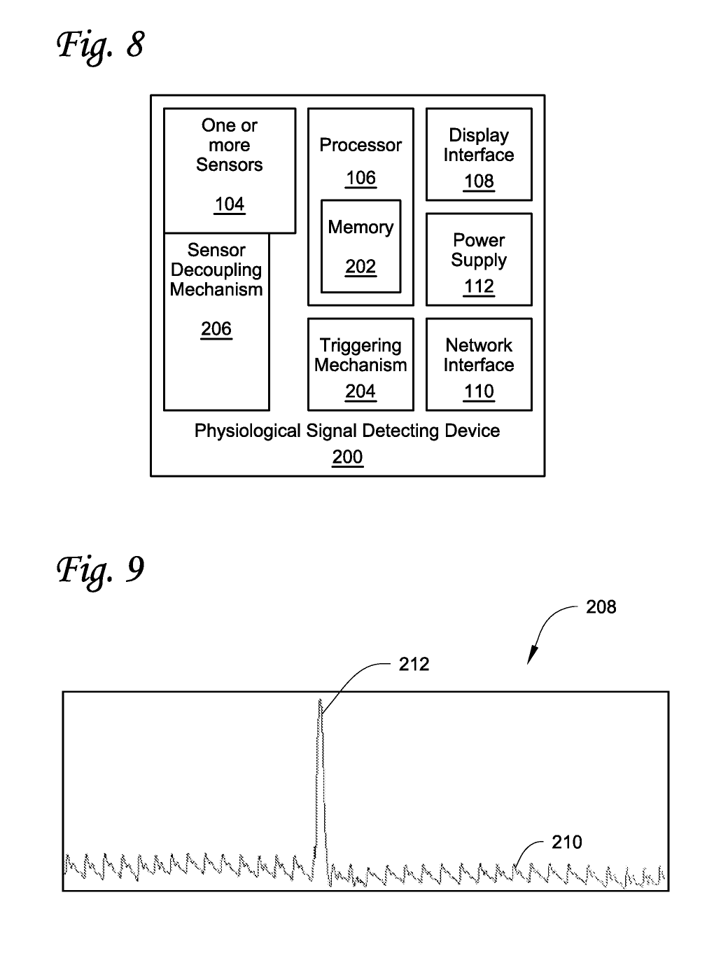

FIG. 8 is a schematic diagram of an embodiment of a physiological signal detecting device 200. The physiological signal detecting device 200 may be substituted for any of the physiological signal detecting devices 102, 126, 132, 136, 144 for respective systems 100, 124, 130, 136, 142 (see FIGS. 1-5). Alternatively, the systems 100, 124, 130, 136, 142 can further include the physiological signal detecting device 200. Similar to the physiological signal detecting devices 102, 126, 132, 136, the physiological signal detecting device 200 includes one or more sensors 104 and a power supply 112. The physiological signal detecting device 200 includes a processor 106, which includes or is connected to a memory component 202 for storing computer readable instructions and/or computer readable electronic data. The processor 106 can also be configured for compression, encoding, decompression, and/or decoding of data. The physiological signal detecting device 200 includes a display interface 108 for providing a user with a means of interacting with the physiological signal detecting device 200. The physiological signal detecting device 200 can also include a network interface 110.

The physiological signal detecting device 200 also includes a trigger mechanism 204. The trigger mechanism 204 is a component separate from the display interface 108. For example, the trigger mechanism 204 may be a button, and/or a reed switch which can be activated with a magnet. Alternatively, the trigger mechanism 204 may be a part of the display interface 108. The trigger mechanism 204 can activate one or more sensors 104 located on a wrist of a person through one or more actions of a hand, fingers, wrist joint, etc. For example, hand movements (e.g., making a fist, flexing one or more fingers, extending one or more fingers, bending the hand back at the wrist, bending the hand forward at the wrist, doing the aforementioned actions at various frequencies, speeds, forces, etc.) can be used to communicate with one or more sensors 104, wherein the one or more sensors 104 is configured with a pressure sensor, a force sensor, a strain gauge, an accelerometer, and/or combinations thereof, so that the one or more sensors 104 can detect the hand movements. The hand movement may have different uses and/or meanings, based on the processor 106 being configured to receive data related to the hand movements from the sensors 104 and interpret the data. Various functions that can be achieved via the hand movements include, for example, activation/deactivation of an auxiliary device, adjustment of data (e.g., change volume of the auxiliary device, etc.), menu navigation of a user interface, etc. The display interface 108 may be used to set various thresholds of the sensor 104 for the detected sensor signal to have a meaning for the processor 106 to carry out a particular function. The particular function to be carried out may be configured via the display interface 108 and stored to the memory component 202. Then, when the sensor 104 detects a signal that meets the threshold criteria, the processor 106 is configured to carry out the particular function associated with the threshold criteria.

The trigger mechanism 204 can detect various triggers intended by the wearer for triggering the detecting device 200 and/or an auxiliary system connected via the network interface 110. The trigger mechanism 204 allows generation of triggered data when certain criteria are met (e.g. one or more physiological parameters surpass a threshold, etc.). When certain criteria are met, the trigger mechanism 204 may automatically initiate the physiological signal detecting device 200 to produce a time-stamped marker (and stored as electronic data to the memory component) and/or initiate collection of data from one or more sensors 104 for a particular time period. The trigger mechanism 204 may also be included in the physiological signal detecting devices 102, 126, 132, 136 and/or the auxiliary devices 114, 128, 134, 140 shown in FIGS. 1-4.

The physiological signal detecting device 200 may provide instructions to the user via the display interface 108 when particular levels of the physiological parameters are determined by the processor 106. For example, the trigger mechanism 204 may be activated when the person being monitored goes to sleep, wakes up during the night, and/or wakes up in the morning. The data related to the physiological signal, user input data, and data related to the interaction with the trigger mechanism 204 may be used by the processor 106 to create a processed data. The processed data may include an index of a continuous scale (e.g., from 0 to 10, from 0 to 100, etc.) indicating a likelihood that a particular disorder is present (e.g., sleep apnea, restless legs, etc.) or the quality of sleep. The processed data may include a status data, which is a qualitative indication of the sleep quality (e.g., letter grade A thru F; 1-5 stars; poor, fair, average, good, excellent; etc.) of the monitored person.

The processed data may be used by the processor 106 to provide feedback information. The feedback information may be provided as an alert, an alarm, a messages, etc. For example, the user may be provided with feedback information which includes information alerting the user to seek medical attention, or to adjust a particular behavior, etc. The feedback information may take the form of a light or message or other visual indicator on the display interface 108 of the physiological signal detecting device 200, and/or on an auxiliary system that is in communication with the physiological signal detecting device 200. The feedback information may be delivered to the user via a physical feedback (e.g., a vibration, a pressure, etc.). For example, the processor 106 can initiate a vibrational feedback via a feedback mechanism to alert the user who suffers from narcolepsy when he or she falls asleep during the day. The feedback information may include instructions for the user, a monitoring administrator, or other person. For example, the user with a particular sleep disorder may be instructed to adjust his medication or to initiate certain relaxation techniques, etc. The processed data and/or feedback information may be communicated to the monitored person, the monitoring administrator, and/or other persons. For example, data related to time to bed, time awake, daily sleep duration, number of arousals, sleep quality index, trends of each, etc. may be provided. The feedback information can be provided by the physiological signal detecting device 200 and/or by one or more auxiliary devices. The physiological signal detecting device 200 and/or an auxiliary device in communication with the physiological signal detecting device 200 may provide feedback information to another system (e.g. implantable or external electrical neurostimulator for apnea, continuous positive airway pressure (CPAP), etc.) to automate changes in its output which may affect the user. For example, if the physiological signal detecting device 200 and/or the auxiliary device determines that a particular event is likely to happen (e.g. apnea event, etc.), either the physiological signal detecting device 200 and/or the auxiliary device may provide feedback information to the auxiliary system, so that the auxiliary system changes its output (e.g., turn on, turn off, changes amplitude, change duration, change frequency, change dosage of drug/stimulation therapy, etc.).

An embodiment of the system may be composed of multiple physiological signal detecting devices 200 such that a user and/or multiple people can cooperate to aid in managing their sleep. For example, different teams of persons trying to better manage their sleep may be formed through an online social network. The teams can then compete for rewards, points, prizes, coupons, etc. Performance of persons and teams may be displayed to show how one team ranks against another and to show how persons on the teams are performing.

An embodiment of the device and/or system provides feedback information to one or more persons to motivate the one or more persons to progress closer to a goal, or to achieve another more desired state. For example, a predictive alert may be communicated to a monitored person to warn him or her that he or she is about to have a panic attack and that he or she should intervene with a panic reducing activity (e.g. controlled breathing, vagal massage, discontinuation of certain activities, changing environment, cognitive behavior therapy, taking a medication, stress reduction method, etc.). In another example, an alert may be communicated to a monitored person to warn him or her that his or her stress exceeds a particular threshold, and that he or she should perform stress reducing activity. In another example, a predictive alert may be communicated to a monitored person (and/or a health professional) that he or she is about to have an epileptic attack or a seizure, so that the person and/or the health professional can take precautionary measures accordingly. In another example, exercise goals (e.g. heart rate, energy burned, etc.) may be communicated to one or more persons to motivate the one or more persons to expend more or less effort to meet a training routine or win a competitive exercise game.

The physiological signal detecting device 200 also includes a sensor decoupling mechanism 206. The sensor decoupling mechanism 206 is connected to and/or is a part of the one or more of the sensors 104. Accordingly, the sensor decoupling mechanism 206 may be included in the physiological signal detecting devices 102, 126, 132, 136 (see FIGS. 1-4).

Detecting physiological signals can be difficult given that the sensors 104 detect not only physiological signals, but also noise due to various movements, motions, and other forces applied to the sensors 104. FIG. 9 is an example of a graph 208 showing a pulse waveform 210 illustrated from pulse signals. The pulse waveform 210 can be provided by detecting and recording the pressure or flow through the ulnar artery and/or the radial artery at the wrist. The detection of the pulse (a physiological signal) can be made with one or more of a pressure sensor, an ultrasound, an impedance sensor, an infrared sensor, an optical sensor, a force sensor, a strain gauge, etc. The graph 208 shows a noise signal 212 (which in this example is an artifact amplitude) caused by an interference motion while the pulse signals were being detected. Accordingly, the noise signal 212 can drown out the physiological signals and/or cause an erroneous determination of physiological parameters that are derived from this data set.

The noise signal 212 can be caused by, for example, when detection of pulse at a person's wrist is disrupted by flexing wrist muscles through the extension and flexion of the fingers (opening the hand and making a fist), finger motion (e.g. typing), bending at the wrist, and/or rotating the hand/forearm. The disruption caused by these various motions and positions by the user can shift the statistical mean signal measurement, change the physiological signal amplitude, and add noise which can be confused with the pulse signal. Changes in signal amplitude resulting from position or motion artifact can cause errors determining physiological parameters from the detected physiological signals. For example, determining a blood pressure parameter from the pulse signals may not be accurate due to the above described artifacts in the detected pulse signals. For example, noise caused by movement may be introduced as an artificial physiological signal, which can mistakenly be determined to be a part of the detected physiological signals. Physiological parameters derived from such physiological signals would be inaccurate. In order to compensate for these errors and inaccuracies, an alternative may be to increase the number of readings and/or increase the physiological signals detected. However, such compensations would necessarily lead to undesirable results of taking longer time to detect the physiological signals and/or require more power (which can lead to shorter useful battery time and/or require a greater power source). The sensor decoupling mechanism 206 can overcome the above disadvantages by reducing and/or eliminating noise caused by the user's movement.

An embodiment of the sensor decoupling mechanism includes soft/conformal portions which can be in series or in parallel with rigid and/or flexible portions (e.g. between rigid portion 230 and skin surface over radius 220 bone or between the bone and the dorsal side of the wrist) to help the sensor decoupling mochas fit and conform to the body, a surface area connected to flexible portions which can maximize contact with sensed area and minimize contact with non-sensed area, a rigid portion for being held against a person's body by one or more flexible/stretchable portions. The flexible/stretchable portion can be separated by intermediate rigid portions and may have alternating sections with orthogonal orientations to minimize transmission of force to the sensor. The sensor decoupling mechanism is configured for avoidance of muscles and tendons when worn, and has an opening inside the band for muscle to expand into. Further, the sensor decoupling mechanism can provide a downstream pressure applied to an artery, which can improve consistency of radial pulse when a hand is rotated. The sensor decoupling mechanism can provide a downstream pressure applied to an artery, and act to expand the artery under the sensor and improve consistency of radial pulse when the hand is moved and especially when it is rotated.

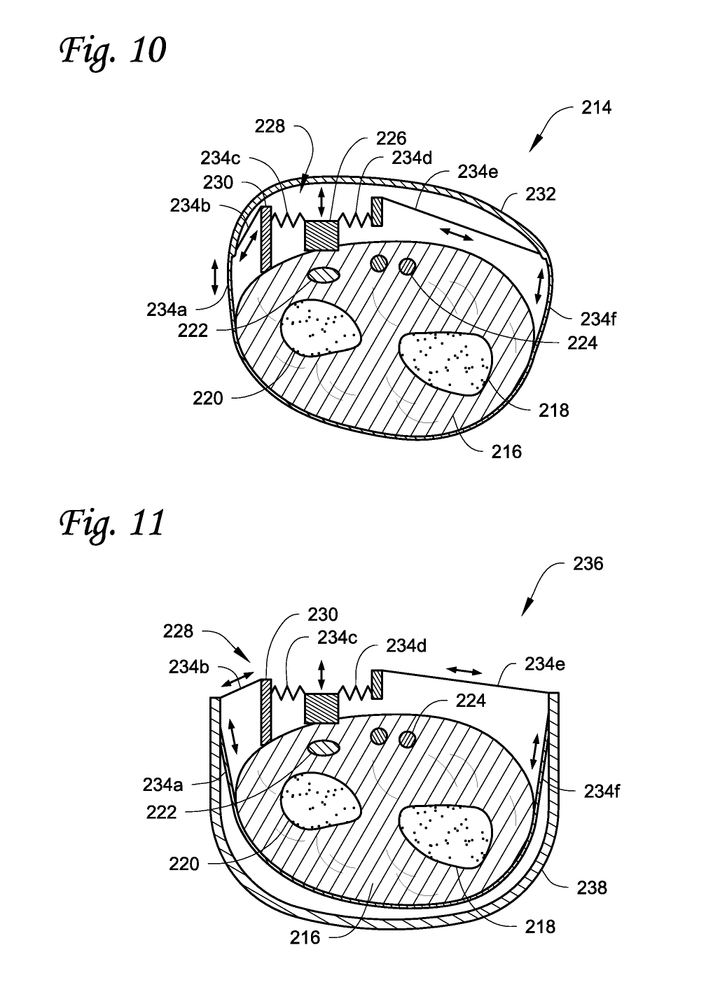

FIGS. 10 and 11 are cutaway side views of embodiments of physiological signal detecting devices. The physiological signal detecting device shown in FIG. 10 is similar to the embodiment of the device shown in FIG. 11. Accordingly, the same reference numbers have been used for the same or similar features.

FIG. 10 is a cutaway side view of the positioning of the physiological signal detecting device 214 with respect to the person's ulna 218, radius 220, radial artery 222, and tendons 224 of the wrist 216. A pulse sensor 226 (e.g., a pressure sensor), which can be one of the sensors 104 described above (see FIGS. 1-4 and 8), is positioned substantially near, above, and/or covering the radial artery 222. Accordingly, the pulse sensor 226 can detect a pulse signal of the person by detecting the blood flow through the radial artery 222. The pulse sensor 226 includes and is connected to an embodiment of a sensor decoupling mechanism 228, which isolates the pulse sensor 226 from movement of other parts of the device 214. The sensor decoupling mechanism 228 includes a rigid portion 230 which, when positioned appropriately, presses on to an outer surface of the wrist near, but not affecting or minimally affecting, the radial artery 222. An example of minimally affecting means less than 10% of the load is applied over the radial artery. The rigid portion 230 can be positioned lateral to the radial artery 222. The rigid portion 230 bears most of the attachment forces and extraneous forces on the pulse sensor 226, so that the pulse sensor 226 responds in a linear motion to the blood flowing through the radial artery 222, moving independently from the rest of the physiological signal detecting device 214. The sensor decoupling mechanism 228 includes a rigid section 232 and a plurality of flexible sections 234a, 234b, 234c, 234d, 234e, 234f. The flexible sections 234a, 234b, 234c, 234d, 234e, 234f may be mechanical springs, elastomers, and/or other compressible/stretchable materials. The rigid section 232 is configured to be on the volar side of the wrist, extending from one side to another side of the wrist. The rigid section 232 is connected to the flexible sections 234a and 234f, which extend along the dorsal side of the wrist 216, securing the physiological signal detecting device 214 to the wrist 216. Between the rigid section 232 and the wrist 216, the rigid portion 230 is connected to the rigid section 232 via flexible section 234b. The flexible section 234e is connected to the rigid section 232. The flexible sections 234c and 234d are connected to the pulse sensor 226. The rigid section 232, the flexible sections 234a, 234b, 234c, 234d, 234e, 234f, and the rigid portion 230 are directly or indirectly connected to the pulse sensor 226 to kinetically and/or kinematically isolate the sensor 226 from the movements of the wrist 216 and/or other parts of the device 214. The rigid section 232, the flexible sections 234a, 234b, 234c, 234d, 234e, 234f, and the rigid portion 230 can be configured to the pulse sensor 226 to kinetically and/or kinematically dampen the forces due to the movements of the wrist 216 and/or other parts of the device 214.

FIG. 11 is a cutaway side view of the positioning of the physiological signal detecting device 236 with respect to the person's ulna 218, radius 220, radial artery 222, and tendons 224 of the wrist 216. A pulse sensor 226, which can be one of the sensors 104 described above (see FIGS. 1-4 and 8), is positioned substantially near, above, and/or covering the radial artery 222. Accordingly, the pulse sensor 226 can detect a pulse signal of the person by detecting the blood flow through the radial artery 222. The pulse sensor 226 includes and is connected to an embodiment of a sensor decoupling mechanism 228, which isolates the pulse sensor 226 from movement of other parts of the device 236. The sensor decoupling mechanism 228 includes a rigid portion 230 which, when positioned appropriately, presses on to an outer surface of the wrist lateral to the radial artery 222, and minimally affecting a blood flow through the radial artery 222. The rigid portion 230 bears most of the attachment forces and extraneous forces on the pulse sensor 226, so that the pulse sensor 226 responds in a linear motion to the blood flowing through the radial artery 222, moving independently from the rest of the physiological signal detecting device 236. The sensor decoupling mechanism 228 includes a rigid section 238 and a plurality of flexible sections 234a, 234b, 234c, 234d, 234e, 234f. The rigid section 238 is configured to be on the dorsal side of the wrist, extending from one side to another side of the wrist. The rigid section 238 is connected to the flexible sections 234a and 234f, which extend along the dorsal side of the wrist 216, between the wrist 216 and the rigid section 238 securing the physiological signal detecting device 236 to the wrist 216. The rigid portion 230 is connected to the rigid section 238 via flexible section 234b and/or 234e. The flexible section 234e is connected to the rigid section 238. The flexible sections 234c and 234d are connected to the pulse sensor 226. The rigid section 238, the flexible sections 234a, 234b, 234c, 234d, 234e, 234f, and the rigid portion 230 are directly or indirectly connected to the pulse sensor 226 to kinetically and/or kinematically isolate the sensor 226 from the movements of the wrist 216 and/or other parts of the device 236. The rigid section 238, the flexible sections 234a, 234b, 234c, 234d, 234e, 234f, and the rigid portion 230 can be configured to the pulse sensor 226 to kinetically and/or kinematically dampen the forces due to the movements of the wrist 216 and/or other parts of the device 236. A difference between the device 236 and the device 214 is the positions of the rigid sections 232, 238. In the device 214 shown in FIG. 10, the rigid section 232 is positioned above the volar side of the wrist 216. In contrast, the device 236 has a rigid section 238 is positioned above the dorsal side of the wrist 216.

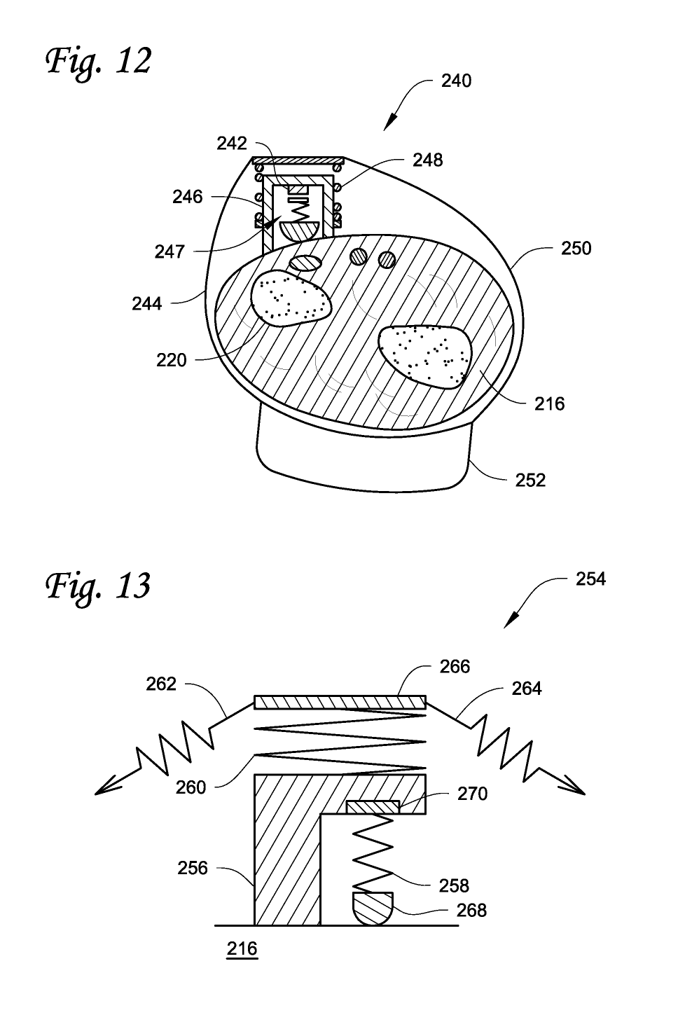

FIG. 12 is a cutaway side view of the positioning of the physiological signal detecting device 240 with respect to the person's ulna 218, radius 220, radial artery 222, and tendons 224 of the wrist 216. A pressure sensor 242, which can be one of the sensors 104 described above (see FIGS. 1-4 and 8), is positioned substantially near, above, and/or covering the radial artery 222. Accordingly, the pressure sensor 242 can detect a pulse signal of the person by detecting the blood flow through the radial artery 222. The pressure sensor 242 includes and is connected to an embodiment of a sensor decoupling mechanism 244, which isolates the pressure sensor 242 from movement of other parts of the device 240. The sensor decoupling mechanism 244 includes a rigid portion 246 which, when positioned appropriately (e.g., above the radius bone), presses on to an outer surface of the wrist near, but not affecting, the radial artery 222. The rigid portion 246 bears most of the attachment forces and extraneous forces on the pressure sensor 242, so that the pressure sensor 242 responds in a linear motion to the blood flowing through the radial artery 222, moving independently from the rest of the physiological signal detecting device 240. The sensor decoupling mechanism 244 includes a flexible inner spring (or material) 246 connected to the rigid portion 246, which allows the inner spring 247 to contract and expand linearly independent of the rigid portion's 246 movement, if any. The rigid portion 246 is held to the surface of the wrist 216 by an outer spring 248, which is preloaded (compressed) so that the force from the compression is in a direction towards and away from the wrist. This force directed away from the wrist is opposed by a strap 250, which wraps around the wrist 216 to secure the rigid portion 246 to the wrist 216. Thus, the compressed outer spring 248 pushes the rigid portion 246 towards the wrist 216. This prevents the rigid portion 246 from substantial movement, even when the wrist 216 is moved and/or flexed. Therefore, the sensor decoupling mechanism 244 prevents movement of the wrist 216 from affecting the physiological signal detected by the pressure sensor 242. The physiological signal detecting device 240 also includes a housing 252 with electronics (e.g., processor, memory, network interface, power supply, display interface, etc.) positioned on the dorsal side of the wrist 216. The strap 250 of the physiological signal detecting device 240 may be stretchable, and may include a low-modulus elastomer, or have an acceptable spring constant property. The strap 250 may be a series of springs and rigid sections like a stretchable metal band. Further, the outer spring may be a low-modulus elastomer. Further, the inner spring may be a low-modulus elastomer.

The embodiments described herein include the flexible components, and/or springs having a spring constant (k) in a range of k<3 lb./in.

The embodiments described herein include the flexible components, and/or springs having a spring constant (k) in a range of k<1.5 lb./in.

The embodiments described herein can include the elastomer having a % elongation/compression property in a range of % elongation >50% elongation.

The embodiments described herein can include the elastomer having a % elongation/compression property in a range of % elongation >200% elongation.

The embodiments described herein can include the low-modulus elastomer having a modulus range of modulus <800 psi.

The embodiments described herein can include the low-modulus elastomer having a modulus range of modulus <400 psi.

FIG. 13 is a close up view of an embodiment of a pressure sensor section 254 of a physiological signal detecting device (which may be similar to the device 240 shown in FIG. 12). The pressure sensor section 254 is a close up view of the rigid portion 256 and various flexible portions 258, 260, 262, 264, which are parts of the sensor decoupling mechanism 266 (which is similar to the sensor decoupling mechanism 244 shown in FIG. 12). The pressure sensor section 254 includes a contact portion 268 which can be positioned substantially near, above, and/or covering the radial artery of a wrist. On an opposing side of the wrist, the contact portion 268 is connected to a flexible portion 258 (e.g., similar to the inner spring 247 shown in FIG. 12). The blood flow through the radial artery causes the contact portion 268 to move linearly, compressing and/or stretching the flexible portion 258, which in turn transfers the forces and pressures to the pressure sensing component 270. The pre-compressed outer spring 260 is connected to the flexible sections 262, 264 of the strap, which wraps around the wrist 216 to secure the rigid portion 256 to the wrist 216. The pre-compressed outer spring 260 may have a pre-compression range of 0.25 lb. to 2 lb. The pre-compressed outer spring 260 may have a pre-compression range of 0.5 lb. to 1.5 lb. The compressed outer spring 260 pushes the rigid portion 256 towards the wrist 216. This prevents the rigid portion 256 from substantial movement, even when the wrist 216 is moved and/or flexed. Therefore, the sensor decoupling mechanism 266 prevents movement of the wrist 216 from affecting the physiological signal detected by the pressure sensor component 270. Although, the inner spring 258 and the outer spring 260 are positioned to be in a series, parallel configuration of two or more springs to accomplish the same or substantially similar function is possible. The embodiments described herein include the flexible components, and/or springs made of material having a viscoelastic property.

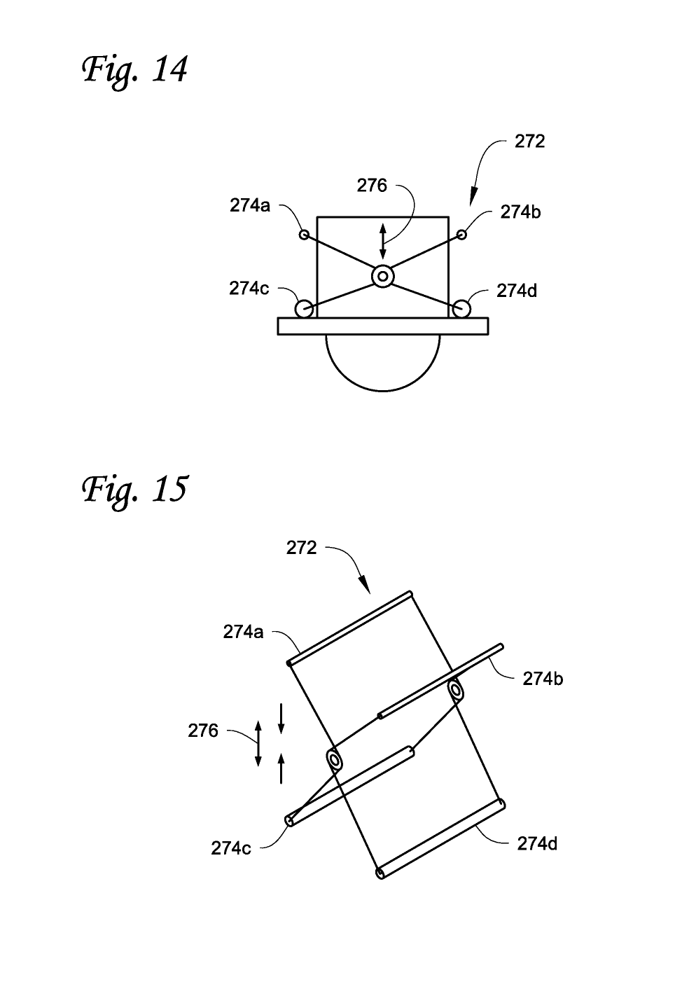

FIGS. 14 and 15 show embodiments of an X-shaped spring 272, which can be used as the outer spring 260 and/or the inner spring 258. The X-shaped spring 272 provides rigidity to prevent tipping and/or buckling. FIG. 14 is a side view, showing force contact sections 274a, 274b and opposing force contact sections 274c, 274d. The force contact sections 274a and 274c move relatively and with each other. The force contact sections 274b and 274d move relatively and with each other. Accordingly, the spring 272 compresses and stretches along a linear direction 276. The parallel surfaces in contact with points 274a, b, c, d move linearly as points 274a, b, c, and d move vertically AND slide outward on these surfaces. The X-shaped spring 272 can occupy a relatively small volume of space, compared to a round wire mechanical spring. The X-shaped spring 272 can also reduce buckling which can be an issue with the round wire mechanical spring. The X-shaped spring 272 can be used in conjunction with a spiral compression spring or elastomer in compression to prevent buckling.

Embodiments of the physiological signal detecting device include a signal processing method for reducing and/or correcting for signal artifacts. The signal processing method can be an alternative to and/or in addition to the sensor decoupling mechanisms described herein.

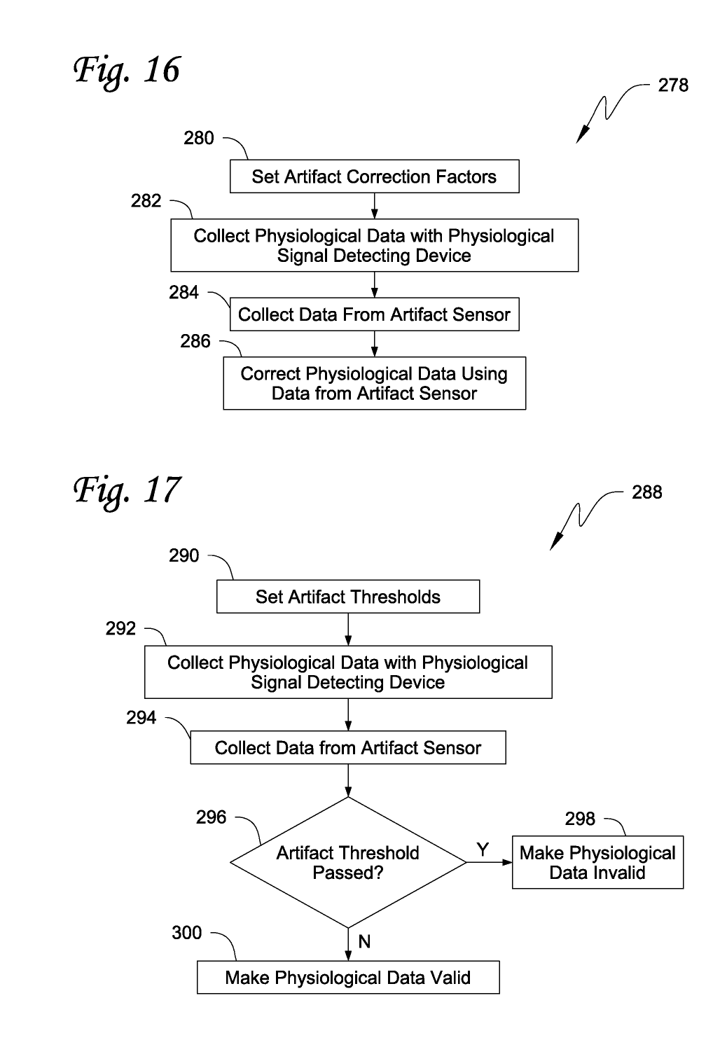

FIG. 16 is a flow chart of an embodiment of the signal processing algorithm for a process 278 in computer readable instructions which is stored and executed by a processor (e.g., the processor 106 shown in FIGS. 1-3 and 8, and/or the processor 118 shown in FIGS. 1-2 and 4). The process 278 uses two or more sensors (e.g., sensors 104 in FIGS. 1-4 and 8). One of the sensors is designated to be an artifact sensor. The process 278 includes a user setting artifact correction factors 280, such as, setting the artifact signal threshold and/or other parameters related to suppressing artifact signals. For example, the artifact signal sensor may be set to ignore all signals having an amplitude of X, when it is assumed that the detected physiological signal data will have amplitudes below X. Accordingly, the artifact signal sensor will disproportionately detect events that are not related to a physiological condition. Alternatively, the artifact signal sensor will only detect events that are not related to a physiological condition. The process 278 includes detecting a physiological signal with the physiological signal sensor 282 and storing the physiological signal as data to a memory. The process 278 includes detecting artifact signal from the artifact sensor 284 and storing the artifact signal as data to the memory. Then, the process 278 includes signal processing 286 of the data using the stored physiological signal data and the artifact signal data. For example, the artifact signal data may be subtracted from the physiological signal data, after the two data are time-matched.

FIG. 17 is a flow chart of an embodiment of the signal processing algorithm for a process 288 in computer readable instructions which is stored and executed by a processor (e.g., the processor 106 shown in FIGS. 1-3 and 8). The process 288 can be performed with a single sensor. However, more than one sensor may be used as well. The process 288 includes a user setting artifact correction factors 290, such as, setting the artifact signal threshold and/or other parameters related to suppressing artifact signals. The process 288 includes detecting a physiological signal with the physiological signal sensor 292 and storing the physiological signal as data to a memory. The process 288 includes detecting artifact signal from the artifact sensor 294 and storing the artifact signal as data to the memory. The processor determines whether the detected artifact signal is greater than or lesser than the pre-set artifact threshold 296. If the detected artifact signal is greater than the artifact threshold, then the processor invalidates and/or removes the physiological signal that corresponds to the artifact signal 298. For example, the invalidated physiological signal may be at the same time or a range of time to the detected artifact signal. If the detected artifact signal is less than the artifact threshold, then the processor validates and stores the physiological signal to a memory 300.

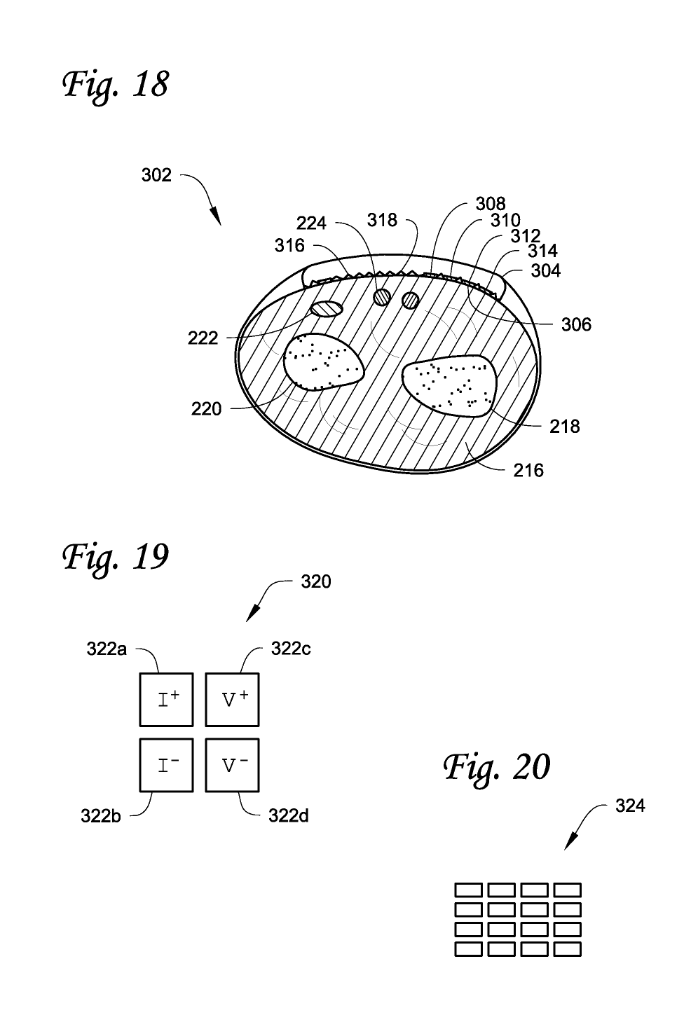

FIG. 18 is a cutaway side view of the positioning of the physiological signal detecting device 302 with respect to the person's ulna 218, radius 220, radial artery 222, and tendons 224 of the wrist 216. A multi-point electrodermal activity sensor 304, which can be one of the sensors 104 described above (see FIGS. 1-4 and 8), is positioned in contact with a surface of a skin 306 at the wrist 216. The multi-point electrodermal activity sensor 304 detects and measures sympathetic responses. Accordingly, the multi-point electrodermal activity sensor 304 can measure stress being experienced by a monitored person. Although using two direct current (DC) electrodes in the multi-point electrodermal activity sensor 304 is possible, detecting galvanic skin response with only two DC electrodes suffers from various problems in real-world applications. The quality of detected signals is affected by the quality of the contact the DC electrodes have to the skin, which can change over time as the monitored person moves, either voluntarily or involuntarily. Further, moisture on the skin, such as sweat, can greatly affect the quality of data. Even when only one of the two electrodes is affected by loss of skin contact and/or moisture on the skin, the data that can be collected via only two DC electrodes is significantly degraded.

For example, for measuring skin impedance, there is a large reduction of impedance for the first 10-20 minutes and so one must wait during this time for the skin to get sufficiently moistened to detect the electrodermal activity. This wait time is worse for DC and gets increasingly better with higher frequency AC because high frequency AC is better able to penetrate the dry cell walls of the skin. The only current paths through the skin for the DC and low frequency AC are the sweat pores so one must wait for the sweat to build up sufficiently prior to measuring electrodermal activity with these methods. Because a four-electrode measurement can provide a constant current drive no matter what the skin impedance is, this delayed effect is removed and one can begin capturing electrodermal activity measurements much sooner.

With only two electrodes, the majority of the measured impedance is the skin-electrode impedance. Thus, the primary measure is the amount of sweat buildup between the electrode and superficial skin and not the activation of the sweat glands. However, because the surface impedance is removed from the four-point electrode, the 4-point electrodermal activity sensor can measure changes to the live skin tissue and thus is a more direct measure of sweat gland activation and sweat within the ducts, whether or not the sweat reaches the surface of the skin.

Further, because it takes time for the sweat to dissipate under the two-electrode DC sensor, there is a hysteresis effect. This is exacerbated when the electrode surface areas are made larger and/or gels are added to reduce the electrode-skin impedance. Larger electrode surface areas and/or gels can increase the amount of sweat trapped between the electrode and the skin, which leads to measurement of sweat accumulation, and not a physiological signal (or physiological parameter). In the four-point electrodermal activity sensor, constant current drive is maintained even if there is high electrode impedance. Thus, individual electrodes do not need to be designed with large surface areas, and do not require gels.

The multi-point electrodermal activity sensor 304 includes multiple electrodes 308, 310, 312, 314. At least two of the electrodes are configured to drive alternating current (AC) through the skin, and at least two of the electrodes are configured to detect the voltage drop through the skin layer. An advantage of AC electrodes is that there is no influence from electrode bias potentials or electrode polarization.

An embodiment of the multi-point electrodermal activity sensor 304 is a four-point electrodermal activity sensor, including two AC driving electrodes 308, 310 and two voltage detecting electrodes 312, 314. An embodiment of the multi-point electrodermal activity sensor 304 includes four or more electrodes or electrode contacts. An embodiment of the multi-point electrodermal activity sensor 304 includes more than four electrodes (or electrode contacts). The two voltage detecting electrodes 312, 314 are configured to measure an impedance in the dermis and/or the living epidermis, for example, about the top 0.5-4 mm, or 0.5-2 mm of skin 306. The four-point impedance sensor may be configured to measure the conductance in the skin 306 and to characterize the electrodermal activity of the sweat glands and/or ducts, in particular the eccrine sweat glands and/or ducts.

The electrodes 308, 310, 312, 314 may be made of one or more materials, rough structure, spikes, and/or coatings including but not limited to: silver, silver-silver chloride, platinum, platinum-iridium, 90-10 platinum-iridium, 80-20 platinum-iridium, nickel, stainless steel, MP35N, conductive carbon, conductive polymers, conductive textiles, conductive materials that maintain a stable potential, etc.

The electrodes 308, 310, 312, 314 have skin contacting surface shapes, including but not limited to: circular, elliptical, oval, rectangular, square, triangular, polygonal, oblong, annulus, etc. Further, one or more corners of the electrodes 308, 310, 312, 314 may be radiused.

In other embodiments, multiple electrodes may be positioned in a pattern including but not limited to: linear, rectangular, square, parallelogram, circular, etc. More than four electrodes may be utilized (e.g., 6, 8, 10, etc.) and different four-point measurements may be made using different combinations of the electrodes for one or more purposes (e.g., to make redundant measurements, to measure different tissue areas, etc.). The electrodes may switch between supplying current and measuring voltage.

In an embodiment, the electrodes 308, 310, 312, 314 are positioned over tissues underneath the dermis having relatively high resistance (e.g., near the ulna 218, over the ulna 218 or radius 220, or other bone) such that a current field is more concentrated through the dermis and/or the living epidermis and measurement may have increased sensitivity to changes in an amount of fluid in sweat glands and/or ducts.

The physiological signal detecting device 302 may include other sensors, such as for example the pressure sensors for detecting pulse signals shown in FIGS. 10-12, and a skin temperature sensor, an ambient temperature sensor, accelerometers, etc.

When in use to monitor the physiological signals, the electrodes 308, 310, 312, 314 should be in contact with the skin firmly, to have very little or no movement relative to the body surface 306. The electrodes 308, 310, 312, 314 may be positioned against a body surface 306 that is away from major muscles, tendons, ligaments, and/or joints to have limited relative motion, pressure, or flexing.

To increase breathability and moisture vapor transmission rate (MVTR) from the skin surface 306 in the area of the electrodes 308, 310, 312, 314, an inner surface 316 may have structures 318 configured to promote MVTR. For example, the inner surface 316 may have physical structures 318, such as, nubs, projections, ridges, channels, grooves, gaps, holes, recesses, and combinations thereof. The physical structure 318 may include porous structures, or have an absorbent material to promote moisture transfer from the skin 306 to the atmosphere (e.g., wicking moisture from the skin 306).

The physiological signal detecting device 302 can be configured to be worn externally on a part of a body (e.g. hand, wrist, forearm, upper arm, shoulder, chest, thorax, abdominal area, back, upper leg, lower leg, ankle, foot, ear, neck, head, etc.). The physiological signal detecting device 302 can be configured to be inserted into the body so as to be fully or partially in or enclosed by a tissue or body cavity (e.g., in artery, vein, lymphatic system, heart, chest cavity, abdominal cavity, alimentary canal/gastrointestinal tract, nostril, ear canal, skull cavity, brain, spine, spinal cord, mouth, trachea, muscle, fat, bone, cartilage, tendon, ligament, nerve tissue, etc.). For example, electrodernal activity may be measured from immediately beneath the dermis or within the epidermis instead of on the surface of the skin. The physiological signal detecting device 302 can be configured to be implanted into the body so as to be fully or partially in or enclosed by a tissue or body cavity

The physiological signal detecting device 302 can include an attachment mechanism for holding the device 302 to the skin. The attachment mechanism may be a sensor decoupling mechanism described herein. Examples of the attachment mechanism include a strap, a band, an adhesive, a clip, a clothing, etc. The physiological signal detecting device 302 can include a fastening mechanism, such as a hook and loop fastener, a clasp, a snap, etc.

The physiological signal detecting device 302 can include a locking mechanism to lock the physiological signal detecting device 302 on a monitored person. Examples of the locking mechanism include a lock and key, a zip tie, etc.

The physiological signal detecting device 302 can include a processor executing computer readable instructions for determining whether the physiological signal detecting device 302 and/or the electrodes 308, 310, 312, 314 are positioned properly. The electrodes 308, 310, 312, 314 can provide resistance and/or conductance signals to the processor which receives the resistance and/or conductance signals to determine whether the contact with the skin has been properly or adequately made. Further, the physiological signal detecting device 302 may include other sensors for determining whether the physiological signal detecting device 302 has been worn and/or worn properly. Examples of the other sensors include resistance or conductance sensors to measure contact with the skin, force or pressure sensors to detect application against the body, any sensor that measures a particular physiological data (pulse sensor, respiration rate sensor, ECG, etc.), sensors that measure motion (e.g. accelerometer, etc.), sensors that can measure a proximity/contact with a body part (e.g. ultrasonic, infrared, etc.), and sensors that can measure how tightly a band is applied (e.g. strain gauge, pressure sensor, etc.). Each of these sensors may have a particular range for determining whether the physiological signal detecting device 302 is being worn and/or worn properly.

In addition to determining whether the physiological signal detecting device 302 is being worn properly, extra parameters (e.g., skin temperature, ambient temperature, accelerometer, force/pressure sensor measuring force/pressure that sensor is applied to body, strain gauge in wrist band measuring how tightly the band is applied, etc.) that are measured can impact the monitored physiological parameters (e.g., pulse, electrodermal activity, skin temperature). Accordingly, these extra parameters can also be used to change and/or correct one or more of the monitored parameters and not just be used to assess whether the device is being worn properly

Additionally, to prevent the wrist monitor from being applied too tightly, a torque limiter can be included to the physiological signal detecting device 302 so that the band cannot be over tightened. Alternatively, a pressure/force sensor in contact with the wrist or a strain gauge in the band can measure a relevant pressure and when a limit is surpassed the user can be notified by the physiological signal detecting device 302 that the band is too tight or too loose. This would be advantageous for positioning the physiological signal detecting device 302 for measuring pulse and/or electrodermal signal.

The physiological signal detecting device 302 can include a processor executing computer readable instructions for determining tissue impedance (e.g. dermal impedance) using data from one or more of the electrodes 308, 310, 312, 314. The processor can perform a weighted combination of a plurality of the electrodes 308, 310, 312, 314, or a tiered combination of the electrodes 308, 310, 312, 314. The resulting tissue impedance (a physiological parameter) may be utilized in other device algorithms to determine a status, index, or prediction for a particular disease, condition, state, etc.

An embodiment of the physiological signal detecting device 302 includes using one or more frequencies of the AC current (e.g., 20 Hz, 100 Hz, 1 kHz, 10 kHz, 50 kHz, and 100 kHz). The frequencies may vary for one or more of the electrodes 308, 310, 312, 314.

An embodiment of the physiological signal detecting device 302 includes using a time frequency over which the skin impedance is measured (e.g., continuously, every 10 seconds, every minute, every 15 minutes, every 30 minutes, every 1 hour, every day, when triggered, etc.).

An embodiment of the physiological signal detecting device 302 includes using a time duration period over which the skin impedance is measured (e.g., less than 1 second, 1 second, 10 seconds, 30 seconds, 1 minute, 5 minutes, 30 minutes, 1 hour, 1 day, 1 week, continuously, etc.).

An embodiment of the physiological signal detecting device 302 includes using one or more duty cycle over which the skin impedance is measured (e.g., 1%, 10%, 50%, and 100%).

Each of the AC current frequency, time frequency, time duration, and/or duty cycle may be varied among the multiple electrodes, and each of the electrodes may vary the AC current frequency, time frequency, time duration, and/or duty cycle.

FIGS. 19-23 show examples of shapes and arrangement of the electrodes for the multi-point electrodermal activity sensor 304. Although current driving electrodes may be described as (I+) or (I−), it will be understood that for AC electrodes, the polarity of the current being driven via the electrode will change (i.e. flip), so that the (I+) becomes (I−), and (I−) become (I+). Voltage detecting electrodes are described as voltage (V+) and voltage (V−).

In an embodiment of the arrangement of the electrodes for a pinched layer of skin, a set of electrodes is placed on one side of the pinched layer of skin, and another set of electrodes is placed on the other side of the pinched layer of skin, so that at least some of the two sets of the electrodes can measure electrodermal activity across (through) the pinched layer of skin.

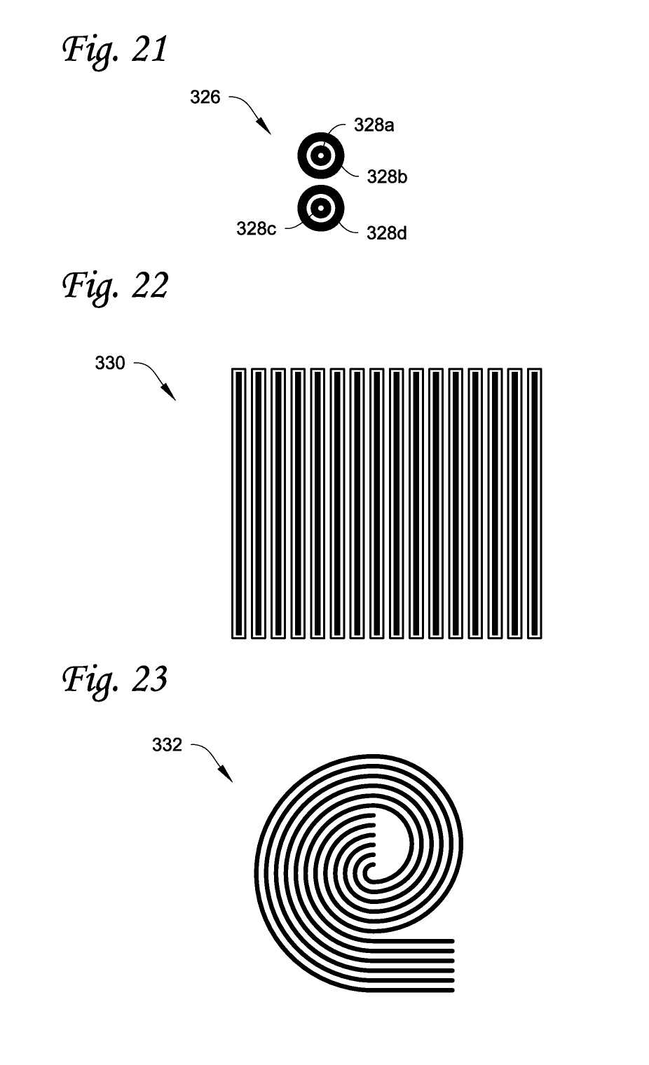

FIG. 19 is a 2×2 matrix arrangement 320 of electrodes 322a, 322b, 322c, 322d for driving current (I+) and current (I−), and detecting voltage (V+) and voltage (V−). FIG. 20 is a 4×4 matrix arrangement of multiple electrodes 324. FIG. 21 is a two ring configuration 326 of four electrodes 328a, 328b, 328c, 328d.

The first set of rings has a detecting electrode for voltage (V+) 328a in the center and a current (I+) driving electrode 328b as a ring around the voltage (V+) electrode 328a. The second set of rings has a detecting electrode for voltage (V−) 328c in the center and a current (I−) driving electrode 328d as a ring around the voltage (V−) electrode 328d. FIG. 22 is a linear arrangement of multiple electrodes 330, wherein the driving electrode for current (I+), detecting electrode for voltage (V+), driving electrode for current (I−), and detecting electrode for voltage (V−) are arranged along a linear direction. The arrangement of the electrodes has a ratio of N+1 current electrodes for every 2N voltage electrodes to allow increased voltage sensing per area so that the sensor can be more sensitive for a given area. FIG. 23 is an arrangement of multiple detectors 332, wherein the voltage (V+), current (I+), voltage (V−), and current (I−) electrodes are positioned alternatingly, then the entire group of the multiple detectors is wound in a spiral configuration. For example, the electrodes may have a repeating pattern of the following alternating arrangement: (I+), (V+), (V−), (I−), (V−), and (V+).

The multiple electrodes can have a center-to-center distance of less than 1 cm.

The multiple electrodes can have a center-to-center distance of less than 5 mm.

The multiple electrodes can have a sufficient center-to-center distance such that the electric field of the current driven through the skin tissue is concentrated primarily superficially in an area of the dermis and/or the living epidermis. Accordingly, a measurement may be more sensitive to changes in the amount of fluid in the sweat glands and/or ducts. The electrodes may be made in a long rectangular, elliptical, oval, oblong shape with the short dimensions arranged in the direction of the current supply such that the centroids of the electrodes may be positioned closely together while still maximizing the electrode surface area available for skin contact. The electrodes may be flexible or may be incorporated into a flexible material (e.g. elastomer band, etc.) such that they conform well to the body surface.

An embodiment of the multi-point electrodermal activity sensor includes a set of two electrode (AC or DC), in addition to a set of the four electrodes (described above). The set of two electrodes can measure skin-electrode impedance for monitoring the overall electrode contact. Data from the two sets of electrodes can be compared for better analysis (or error detection) of the electrodermal activity. For example, error in the data may be caused by an accumulation of sweat in the stratum corneum or between the skin and a particular electrode.

A set of two skin potential electrodes can also be added to an embodiment for measuring skin potential response (SPR). An activation of the sweat glands causes a change to the skin potential in addition to the change in conductance/impedance. This change can be detected between two electrodes, in which one electrode located at the same site as the set of the four-electrode sensor, and an indifferent electrode located elsewhere on the body (e.g., further up the arm or on the opposite side of the wrist as the electrodermal sensor). Some of the physiological parameters which can be measured are, for example, AC impedance, skin impedance level, admittance, phase angle, etc. An impedance measurement can be made at one or more frequency between 20 Hz and 100 kHz. An impedance measurement can be made at one or more frequency below 10 kHz. The frequencies below 10 kHz have more sensitivity to extracellular fluid changes whereas the frequencies above 10 kHz penetrate the cell walls and are more sensitive to intracellular hydration. Different frequencies also can be used to modify the depth of measurement.

The electrolytic conduction in the skin can be affected by an increase in skin temperature whereby the conductivity increases with an increase in temperature and may be approximated by a 2% change per degree C. change. This relationship may follow the Arrhenius equation. The device may use a skin temperature sensor to correct for changes in skin temperature.

Changes in electrode pressure may affect the electrodermal activity sensor. The sensor decoupling mechanism may be used to help mitigate this. For example the pressure of the electrode to the skin could be maintained at a consistent level between 0.25 psi and 4 psi.

As a way of measuring an accumulation of sympathetic activation or sweat gland activation, the embodiments described herein may include a processor having a computer readable and executable instructions having an algorithm that either counts the number of electrodermal response pulses or integrates the cumulative area under the electrodermal response pulses to provide an aggregate measure of sweat gland activation.

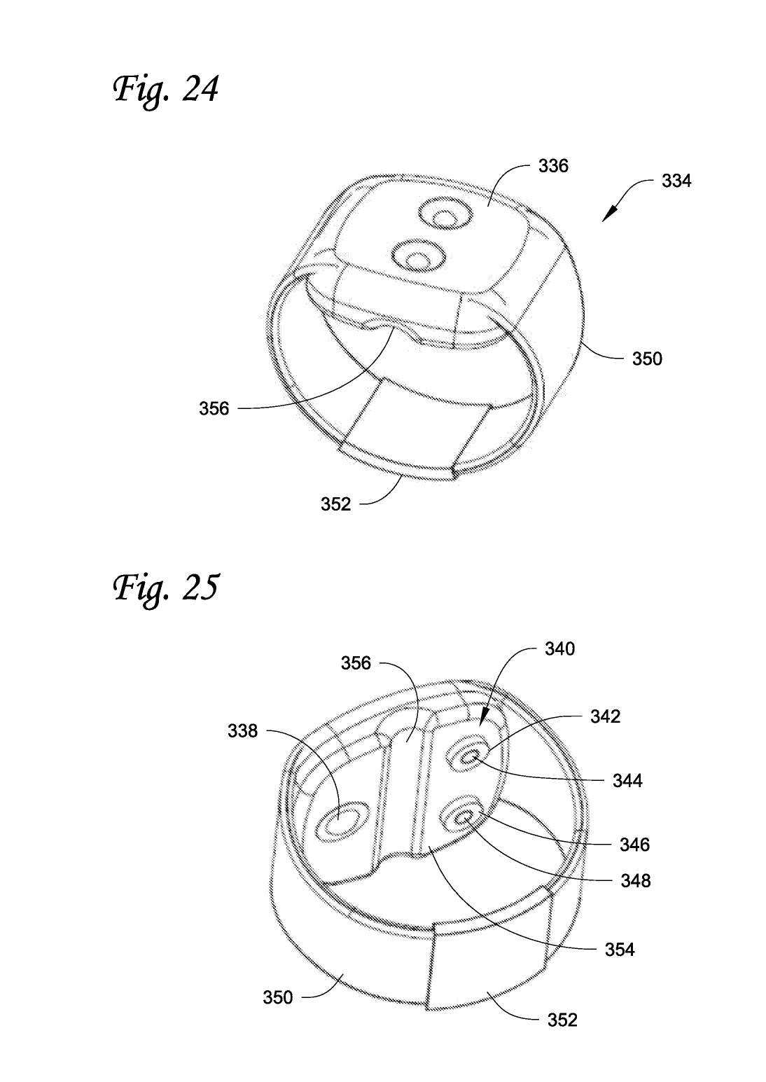

FIG. 24 is an embodiment of a physiological signal detecting device 334. The physiological signal detecting device 334 may be substituted for any of the physiological signal detecting devices 102, 126, 132, 136, 144, 200. The physiological signal detecting device 334 includes a display interface 336.

FIG. 25 is another view of the embodiment of the physiological signal detecting device 334. The physiological signal detecting device 334 includes a pulse sensor 338 having a sensor decoupling mechanism (not shown), and a multi-point electrodermal activity sensor 340 with at least four electrodes 342, 344, 346, 348.

FIGS. 24 and 25 show the physiological signal detecting device 334 having a strap 350 and a locking (and/or fastening) mechanism 352 for securing the physiological signal detecting device 334 to a body part of a mammal.

To increase contact stability the electrodes 342, 344, 346, 348 at the skin surface, an inner surface 354 of the physiological signal detecting device 334 has a channel structure 356 configured to avoid contacting the tendons of the wrist.

Another embodiment of the device and system allows for determining a mammal's identification by detecting physiological signals and then comparing the detected physiological signals to pre-stored physiological parameters. Accordingly, the physiological signals can be a form of biometric, allowing the device and system to perform biometric authentication.

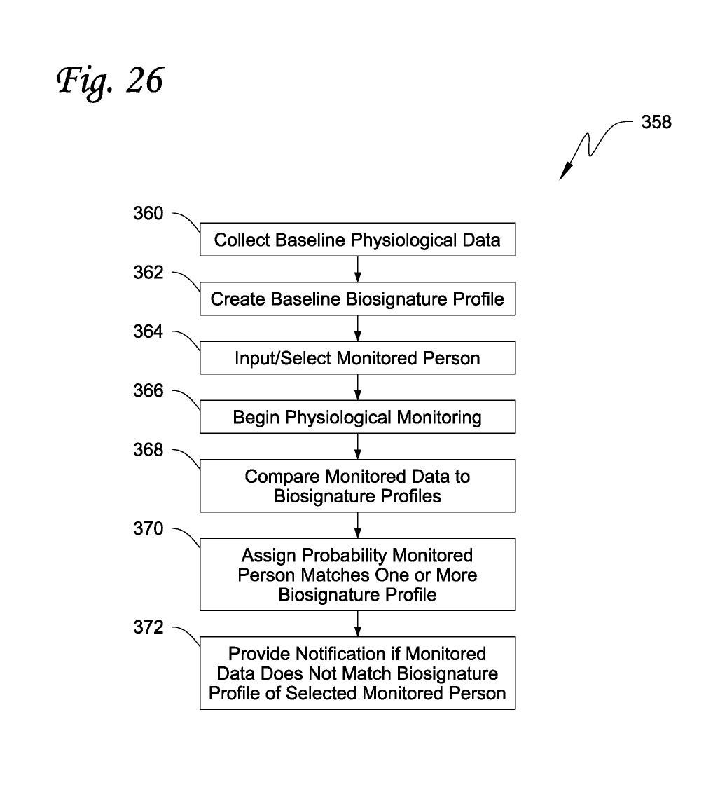

FIG. 26 is a flow chart of an embodiment of an algorithm for a process 358 which can be in computer readable instructions stored and executed by a processor (e.g., the processor 106 shown in FIGS. 1-3 and 8 and/or the processor 118 shown in FIGS. 1, 2, and 4). The process 358 is for determining an identity of a monitored person based on deviations of one or more monitored physiological signals (e.g. skin impedance level, heart rate, heart rate variability, activity profiles, heat flux, etc.). The process 358 includes a step of collecting one or more baseline physiological data and storing that data 360. Then, a baseline biosignature profile (e.g., authentication of identity) is created 362 based on the collected baseline physiological data. Then the process 358 requires that the stored baseline biosignature profile is selected 364 for comparing to physiological data which will be detected. Detecting and/or monitoring of a person's physiological signals is started, and the physiological signals are converted into electronic data and stored to a memory 366. The collected data is then compared to the selected biosignature profile (i.e., baseline physiological data) 368. The processor determines a statistical probability of a match between the collected data and the selected biosignature profile 370. A notification of the probability is provided 372, particularly if there is a high probability that the monitored person's identity does not appear to match that of the biosignature profile.

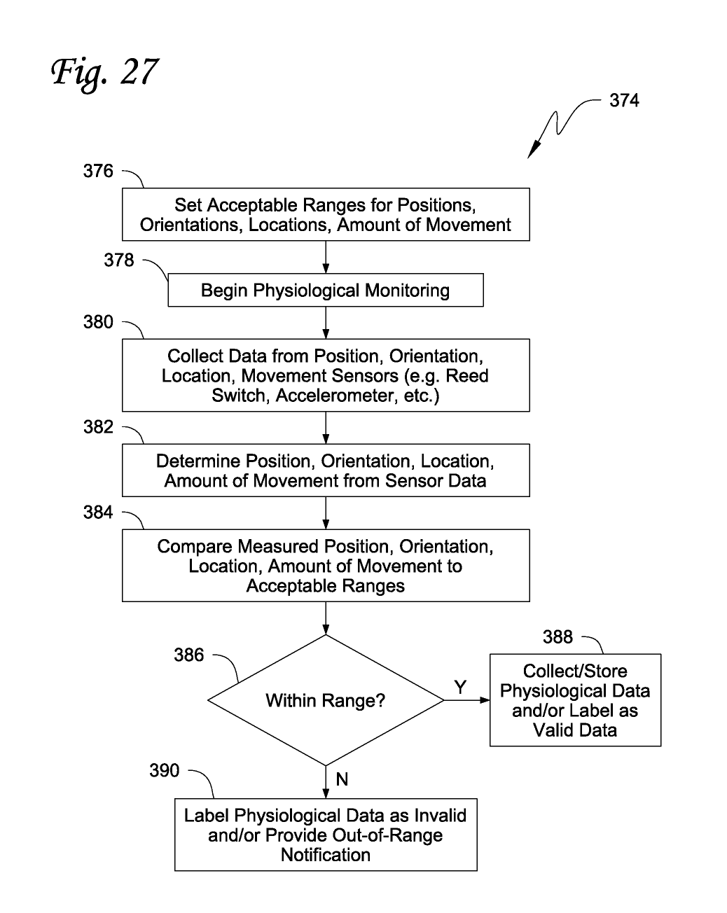

FIG. 27 is a flow chart of an embodiment of an algorithm for a process 374 which can be in computer readable instructions stored and executed by a processor (e.g., the processor 106 shown in FIGS. 1-3 and 8 and/or the processor 118 shown in FIGS. 1, 2, and 4). The process 374 is for ensuring that a physiological signal detecting device is at a particular position, orientation, location and/or that there is limited motion before accepting a physiological data as valid.

For example, determining a valid physiological parameter, such as blood pressure, requires that a limb from which the blood pressure signals are detected from is positioned at or near the level of the person's heart.

The process 374 can automate determining whether the physiological signal detecting device is positioned correctly so that good physiological signals can be gathered for determining a valid blood pressure parameter.

The process 374 includes a step of setting acceptable ranges for one or more positions, orientations, locations, movement, etc. of the physiological signal detecting device 376. The physiological signal detecting device may include a reed switch, which is an electrical switch operable via a magnetic field. Accordingly, the person may position the physiological signal detecting device near a magnet, wherein the magnet has been placed according to a predetermined position which has been determined to be adequate for gathering good physiological signals. For example, the magnet may be positioned on a particular location of the monitored person, or on another device (e.g., handrail on a hospital bed, handle bars on a bike, etc.). The magnet may be attached to an adhesive patch, or worn on clothing. An accelerometer in the physiological signal detecting device may be used to determine a position, orientation, location, and/or movement of the physiological signal detecting device. The accelerometer in the physiological signal detecting device may be used to determine that the position, orientation, location, and/or movement of the physiological signal detecting device is within an acceptable range of possible positions, orientations, and/or locations.

The process 374 includes starting the monitoring 378 the physiological signals. Then, collecting 380 data related to the physiological signals at the position, orientation, and/or location of the physiological signal detecting device. Then, the processor determines 382 the position, orientation, and/or location of the physiological signal detecting device. Then the processor compares 384 the detected and/or determined position, orientation, location, and/or movement of the physiological signal detecting device to the set acceptable ranges. The processor determines 386 whether the position, orientation, location, and/or movement of the physiological signal detecting device is within the set acceptable ranges. If it is acceptable 388, then the physiological signal detecting device begins to detect for physiological signals for determining the physiological parameters. If the position, orientation, location, and/or movement of the physiological signal detecting device is not acceptable 390, then the processor discards any physiological signals detected and/or provides a notification to the user that the position, orientation, location, and/or movement of the physiological signal detecting device is not within the set acceptable ranges.

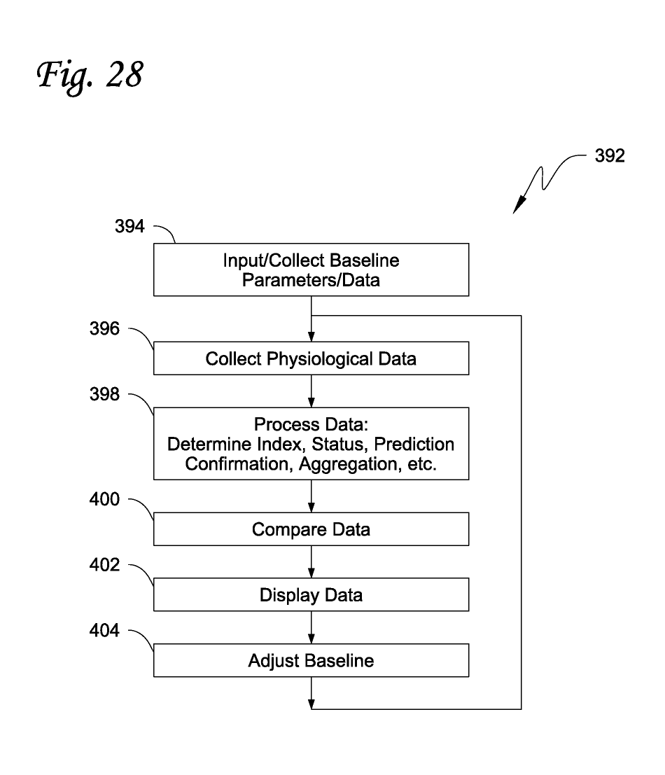

FIG. 28 is a flow chart of an embodiment of an algorithm for a process 392 which can be in computer readable instructions stored and executed by a processor (e.g., the processor 106 shown in FIGS. 1-3 and 8 and/or the processor 118 shown in FIGS. 1, 2, and 4). The process 392 uses one or more physiological data related to physiological parameters to determine and communicate processed data (e.g. averaged data, aggregated data, derived data, an index, status, prediction, etc.) related to one or more of the following conditions: stress, distress, panic, arousal, engagement, excitement, romantic interest, fear, sympathetic tone, parasympathetic tone, health, wellness, exercise performance, weight loss/gain, training performance, gaming performance, triage ranking, honesty/deception, sleep quality, happiness, calmness, improvement or worsening of condition or disease state (e.g. heart failure decompensation, depression, hypertension, etc.). One or more physiological data may serve as a confirmation to one or more physiological data or calculations thereof. Baselines may be determined from a predetermined list, input specific to characteristics (e.g. age, weight, height, race, gender, disease type, performance score, etc.) of a monitored person, an initial physiological data set collected from the monitored person, or a combination of one or more of these determinants.

The process 392 includes input of baseline physiological parameters 394, collecting physiological data by detecting physiological signals using a physiological signal detecting device and/or system 396, and processing data by the processor of the physiological signal detecting device and/or system 398. The processing step 398 includes determining by calculation an index, status, and prediction by using one or more of the aforementioned physiological data in one or more of the following ways: calculating a rate of change of one or more physiological data, variance from a baseline of one or more physiological data, weighted combination of a plurality of physiological data, tiered combination of a plurality of physiological data, verification of physiological data being within an acceptable range, etc. The process 392 includes comparing the processed data to the baseline data 400 and displaying the data and/or results of the comparison 402. The process 392 can adjust the baseline data 404 based on the comparison performed in step 400. The process 392 can determine one or more indices calculated from the accumulation of deviations from a trending data.

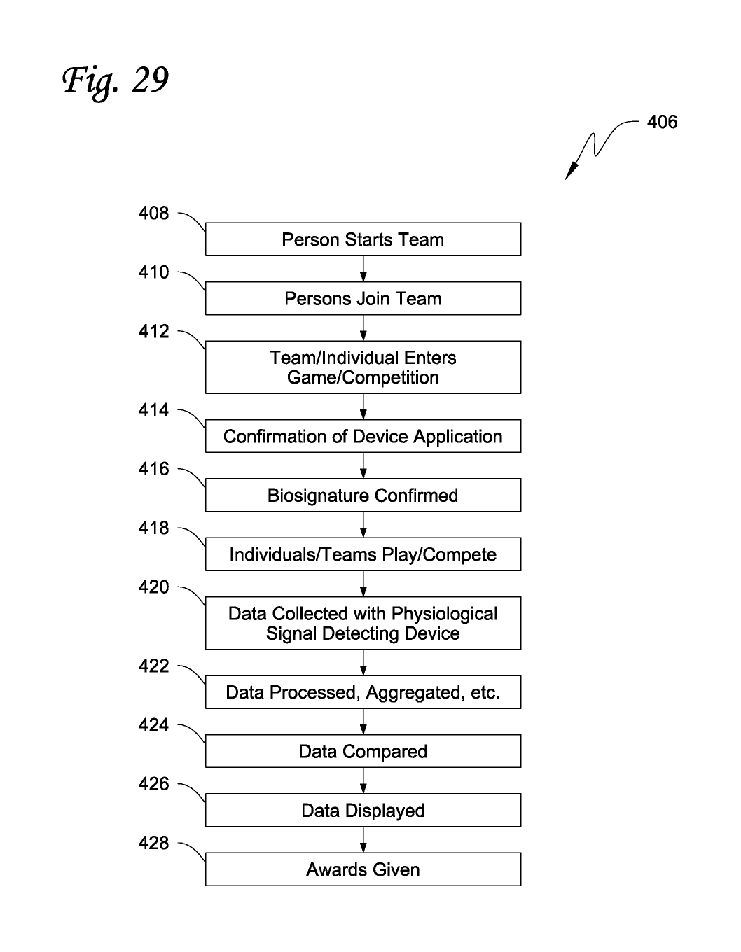

FIG. 29 is a flow chart of an embodiment of a process 406 for using the embodiments of the device and system described herein. The process 406 is for encouraging cooperation and/or competition among people who are being monitored by the physiological signal detecting device and/or system.

The monitored people can compete for various purposes (e.g. video gaming, real-life gaming scenarios, smoking cessation, drug treatment, weight loss, stress reduction, exercise, improved sleep, heart health management, disease management, athletic training, military training, etc.). The monitored people can start a team 408 and/or join a team 410 through an online social network platform. By being on a team, the monitored people may be more motivated to perform (e.g. to lose weight, to reduce stress, etc.) so as not to let team members down. Competition may also help motivate people to perform better.

The process 406 includes a step of a monitored person or a team entering a competition 412. Verification that a proper device application is installed on the device is performed 414. Biosignature verification is performed 416 (see FIG. 26 and description of the process 358 for determining an identity of a monitored person based on biosignature profile). Accordingly, cheating may be suppressed to ensure that the physiological signal detecting device is being worn by an authorized person.

The process 406 includes the monitored people and teams engaging in various activities which may result in various physiological signal outputs 418. These physiological signal outputs are detected and stored 420 as data in a computer readable format to one or more memory. One or more processors process the data 422 and then compare the data 424. The data can be displayed 426 to the people and teams who participated in the activity. The data (e.g., aggregated data and relative performance data of competing people and/or teams) may be displayed on a website, social network, and/or through a local software program and may be shown on one or more physiological signal detecting devices and/or systems. For example, the data may be displayed on a smart phone, on a screen on a stationary bike, and/or projected on a wall in front of an exercise class.

Awards can be given out 428 to the participants for having achieved certain desired performance or for winning competitions to motivate the participating persons. Rewards or points may be accumulated with different timing (continuously, daily, at the end of each competition, etc.). The process 406 can be used during daily life or in a defined setting (e.g. aerobics class, spinning class, athletics practice, military training session, etc.).

With regard to the foregoing description, it is to be understood that changes may be made in detail, especially in matters of the construction materials employed and the shape, size and arrangement of the parts without departing from the scope of the present invention. It is intended that the specification and depicted embodiment to be considered exemplary only, with a true scope and spirit of the invention being indicated by the broad meaning of the claims.

1. (canceled)

2. A physiological signal detecting device, comprising:
a sensor that detects a physiological signal;
the sensor connected to a sensor decoupling mechanism that reduces noise in the physiological signal detected by the sensor; and
a processor that receives the physiological signal from the sensor, and converts the physiological signal to a physiological parameter,
wherein the sensor is configured to be positioned above an artery of a mammal,
the processor is configured to determine the position of the sensor relative to the artery of the mammal and output information regarding the position of the sensor relative to the artery of the mammal based on the received physiological signal, and
the sensor decoupling mechanism includes a rigid portion configured to be positioned at lateral of the artery, wherein the rigid portion minimally affect a blood flow through the artery.

3. A physiological signal detecting device, comprising:
a sensor that detects a physiological signal;
the sensor connected to a sensor decoupling mechanism that reduces noise in the physiological signal detected by the sensor;
a multi-point electrodermal activity sensor having at least two alternating current (AC) driving electrodes, and at least two voltage sensing electrodes; and
a processor that receives the physiological signal from the sensor, and converts the physiological signal to a physiological parameter, and which receives a second physiological signal from the multi-point electrodermal activity sensor, and converts the second physiological signal to a second physiological parameter.

4-17. (canceled)
